(19) United States
(12) Patent Application Publication
(10) Pub. No.: US 2026/0256661 A1
(43) Pub. Date: Sep. 3, 2026

(54) SOLID HYDROGEL STRUCTURE

(71) Applicant: KAO CORPORATION, Tokyo (JP)

(72) Inventors: Kensuke AOYAGI, Cincinnati, OH (US); Adam SCHROTT, Cincinnati, OH (US); Hayato MIYAZAKI, Tokyo (JP); Yukio SETA, Tokyo (JP); Yumi HAYASHIDA, Tokyo (JP); Takahiro KAWAMOTO, Tokyo (JP); Shingo HIRONO, Tokyo (JP)

(73) Assignee: KAO CORPORATION, Tokyo (JP)

(21) Appl. No.: 19/260,765

(22) Filed: Jul. 7, 2025

Related U.S. Application Data
(60) Provisional application No. 63/764,979, filed on Feb. 28, 2025.

(51) Int. Cl. A61K 8/04; A61K 8/06; A61Q 17/04; A61Q 19/00

(52) U.S. Cl. CPC A61K 8/042; A61K 8/062; A61Q 17/04; A61Q 19/007; A61K 2800/412; A61K 2800/413; A61K 2800/48; A61K 2800/805

(57) ABSTRACT

A solid hydrogel structure that has a specific shape and is capable of exhibiting high functionality by being applied to skin and/or hair, and a method of producing the same. The solid hydrogel structure includes: a continuous phase that is formed of a hydrogel containing a water-soluble polymer gelling agent and water; and a dispersed phase that is formed of an emulsion containing an aqueous phase and an oil phase and dispersed in the continuous phase.

### TECHNICAL FIELD

The present disclosure relates to a solid hydrogel structure in which an emulsion is dispersed in a gel matrix and a method of producing the same.

### BACKGROUND ART

An emulsion containing an aqueous phase and an oil phase is used in a variety of topical compositions such as a humectant, a sunscreen agent, and an ointment (e.g., Patent Literatures 1 and 2). Such a topical composition to be applied to skin and/or hair is generally in a form having fluidity and is used by applying an appropriate amount to the skin and/or hair.

### CITATION LIST

### Patent Literature

Patent Literature 1: Japanese Patent Application Laid-open No. 2024-95185
Patent Literature 2: Japanese Patent Application Laid-open No. 2023-129539

### DISCLOSURE OF INVENTION

A solid hydrogel structure according to an embodiment of the present disclosure includes:
a continuous phase that is formed of a hydrogel containing a water-soluble polymer gelling agent and water; and
a dispersed phase that is formed of an emulsion containing an aqueous phase and an oil phase and dispersed in the continuous phase.

A method of producing a solid hydrogel structure according to another embodiment of the present disclosure includes:
a step of generating a gelling agent aqueous solution in which a water-soluble polymer gelling agent is dissolved in water by heating the water-soluble polymer gelling agent and the water to a first temperature range of equal to or higher than a dissolution temperature of the water-soluble polymer gelling agent while stirring them;
a step of generating an emulsion dispersion liquid by adding an emulsion containing an oil phase and an aqueous phase to the gelling agent aqueous solution in a second temperature range of a sol-gel transition point of the water-soluble polymer gelling agent and stirring them;
a step of stopping the stirring of the emulsion dispersion liquid in the second temperature range;
a step of injecting the emulsion dispersion liquid into a mold; and
a step of cooling the emulsion dispersion liquid injected into the mold to a third temperature range of less than the sol-gel transition point.

### BRIEF DESCRIPTION OF DRAWINGS

FIG. 1 is a schematic diagram of a solid hydrogel structure according to an embodiment.

FIG. 2 is a flowchart showing a method of producing a solid hydrogel structure according to another embodiment.

FIG. 3 is a flowchart showing a method of producing a solid hydrogel structure according to still another embodiment.

### DETAILED DESCRIPTION OF THE INVENTION

The present disclosure relates to a solid hydrogel structure that has a specific shape and is capable of exhibiting high functionality by being applied to skin and/or hair, and a method of producing the same.

Embodiments will be described below in detail.

### 1. DEFINITION OF TERMS

In the description of the present disclosure, in the case where numerical limits or ranges are recited, the end points are included unless otherwise specified. As used herein, the terms “comprise (include)” and/or “contain” specify the presence of stated features, integers, steps, operations, elements, and/or components, but do not exclude the presence or addition of one or a plurality of other features, integers, steps, operations, elements, components, and/or groups thereof.

As used herein, when describing the size and/or position, the terms “approximately” or “substantially similar” can be used to indicate that the described value and/or position is within a reasonably expected range of the value and/or position. For example, regardless of the presence or absence of the modifier “approximately”, the numerical values can have values within ±0.1% of the stated value (or range of values), ±1% of the stated value (or range of values), ±2% of the stated value (or range of values), ±5% of the stated value (or range of values), ±10% of the stated value (or range of values), ±15% of the stated value (or range of values), or ±20% of the stated value (or range of values). A parameter having an “approximately” certain value includes the value itself. In the description of the present disclosure, in the case where numerical limits or ranges are recited, the end points are included unless otherwise specified. Further, all values and small ranges within the numerical limits or ranges are specifically included as if expressly stated.

As used herein, the “compound” is intended to refer to a chemical entity regardless of whether or not it is a solid, a liquid, a gas, a crude mixture, or an isolated and purified product.

In the present specification, the expression the “content of B in A” refers to the ratio (mass %) of the mass of B when the total mass of A is 100 mass %. That is, the “content of B in A” refers to the concentration of B relative to the total mass of A.

### 2. SOLID HYDROGEL STRUCTURE

### 2.1 Overall Structure

The solid hydrogel structure according to an embodiment is a structure for locally applying to the surface of skin and/or hair and is also referred to as a “structure” in the present specification. The structure according to an embodiment can be used as a topical composition such as a cosmetic, a medicine, and a quasi-drug and can be suitably used as a cosmetic composition particularly. In one embodiment, the structure can be expressed as a gelled lotion capsule, a melt, a drop, a puck, a slice, a chip, a flake, a gel, a bead, a ball, a bit, a cube, a bar, a pat, a bar, a sliver, a portion, a dollop, or a shaped lotion.

In one embodiment, the structure is molded and cut into any variety of three-dimensional shapes and retains a predetermined structure. In one embodiment, from the viewpoint of ease of handling, the structure has a total volume in a range of favorably 0.125 cm³ or more and 40 cm³ or less, more favorably 0.5 cm³ or more and 20 cm³ or less, still more favorably 0.8 cm³ or more and 10 cm³ or less, still more favorably 1 cm³ or more and 2.5 cm³ or less. The total volume may be in a range of favorably 0.125 cm³ or more, more favorably 0.5 cm³ or more, still more favorably 0.8 cm³ or more, still more favorably 1 cm³ or more, and favorably 40 cm³ or less, more favorably 20 cm³ or less, still more favorably 10 cm³ or less, still more favorably 2.5 cm³ or less. Further, the three-dimensional shape of the structure is not particularly limited and may be a prismatic shape, a columnar shape, another polyhedral shape, a spherical shape or a shape similar thereto, a drop shape, a rod shape, a heart shape, a star shape, a diamond shape, or any other arbitrary shape.

In one embodiment, from the viewpoints of sufficiently retaining the structure and usability, the structure has rupture stress in a range of favorably 10 kPa or more and 65 kPa or less, more favorably 10 kPa or more and 60 kPa or less, still more favorably 15 kPa or more and 60 kPa or less, still more favorably 18 kPa or more and 60 kPa or less. In one embodiment, from the viewpoint of improving usability, the structure rupture stress of favorably 10 kPa or more and 65 kPa or less. The rupture stress may be in a range of favorably 10 kPa or more, more favorably 15 kPa or more, still more favorably 18 kPa or more, and favorably 65 kPa or less, more favorably 60 kPa or less. In addition, it may have rupture stress of 8 kPa or more. Note that the rupture stress of the structure is a value measured by the method described below in Examples.

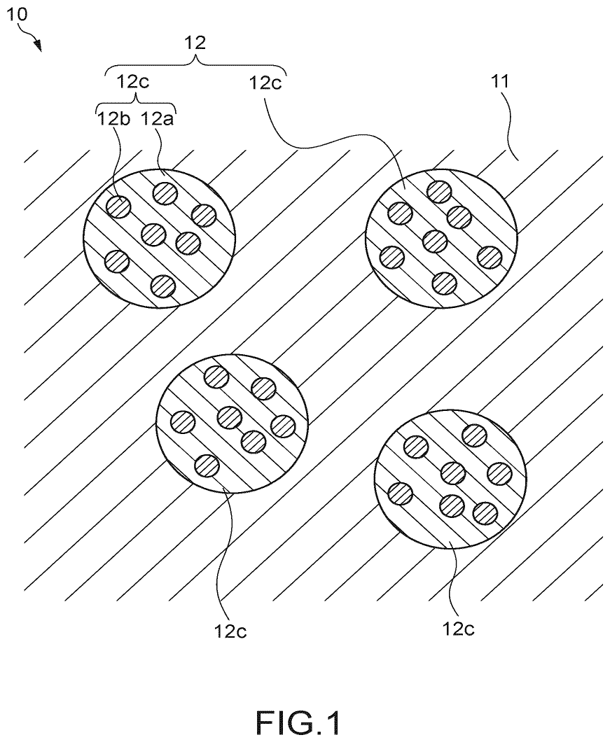

FIG. 1 schematically illustrates a solid hydrogel structure (structure) 10 according to an embodiment. The structure 10 according to an embodiment includes a continuous phase 11 and a dispersed phase 12.

The continuous phase 11 is a continuous phase that is formed of a hydrogel containing a water-soluble polymer gelling agent and water. The continuous phase 11 is formed of a solid gelled product.

The dispersed phase 12 is formed of an emulsion 12c that contains an aqueous phase 12a and an oil phase 12b and dispersed in the continuous phase 11. In one embodiment, the dispersed phase 12 includes a plurality of independent emulsions 12c. The “plurality of independent emulsions 12c” refers to a plurality of droplet emulsions separated by the continuous phase 11. In one embodiment, the emulsion 12c is configured as a functional agent that exhibits a predetermined function when applied to skin and/or hair. Note that the emulsion 12c in FIG. 1 exemplifies an oil-in-water emulsion in which the oil phase 12b is dispersed in the aqueous phase 12a.

As described above, the structure 10 according to an embodiment has a structure in which the dispersed phase 12 formed of the emulsion 12c is dispersed in the solid (gelatinous) continuous phase 11 formed of a hydrogel. The continuous phase 11 being in a fixed shape provides advantages such as excellent portability, suppression of liquid splashing or leakage during use, clear indication of the use amount per application, and superior hygiene as a single package can be used in one application. Further, since the structure 10 can be molded into any three-dimensional structure, it can also be designed to have an appearance that is easy for consumers to pick up.

In addition, since the dispersed phase 12 is retained in the continuous phase 11, it is possible to enhance the functionality (moisturizing properties, a sunscreen function, etc.) of the emulsion 12c of the dispersed phase 12 when applied to skin and/or hair. The reason why the structure 10 according to an embodiment achieves such effects is not clear. However, it is conceivable that when the structure 10 is applied to skin and/or hair, the emulsion 12c in the state of being contained in the gel of the continuous phase 11 is likely to remain at the application site, and the functionality of the emulsion 12c at the application site can be sufficiently exhibited.

Further, from the viewpoint of sufficiently achieving the above-mentioned operation and effect, the structure 10 can take the following configuration. That is, the mass ratio of the dispersed phase 12 to the continuous phase 11 in the structure 10 (the mass of the dispersed phase 12/the mass of the continuous phase 11) is favorably 10/90 or more, more favorably 20/80 or more, still more favorably 30/70 or more, from the viewpoint of reliably achieving the function of the emulsion 12c of the dispersed phase 12, and favorably 90/10 or less, more favorably 80/20 or less, still more favorably 70/30 or less, from the viewpoint of exhibiting the effect of enhancing the functionality of the emulsion by the continuous phase 11.

The content of the dispersed phase 12 in the structure 10 is favorably 10 mass % or more, more favorably 20 mass % or more, still more favorably 30 mass % or more, from the viewpoint of reliably achieving the function of the emulsion of the dispersed phase 12, and favorably 90 mass % or less, more favorably 80 mass % or less, still more favorably 70 mass % or less, from the viewpoint of exhibiting the effect of enhancing the functionality of the emulsion by the continuous phase 11.

The diameter of the dispersed phase 12 in the structure 10 is favorably 0.001 μm or more, more favorably 0.01 μm or more, still more favorably 0.1 μm or more, from the viewpoint of reliably achieving the function of the emulsion of the dispersed phase 12, and favorably 1000 μm or less, more favorably 100 μm or less, still more favorably 50 μm or less, from the viewpoint of improving the separation stability. Note that the diameter of the dispersed phase 12 is a volume average particle diameter of the particle of the independent emulsion 12c.

In one embodiment, the content of water in the structure is in a range of 10 mass % or more and 98 mass % or less, favorably 25 mass % or more and 96 mass % or less, more favorably 35 mass % or more and 95 mass % or less, still more favorably 55 mass % or more and 94 mass % or less, still more favorably 65 mass % or more and 92 mass % or less, or the amount necessary for making the total mass of the components equal to 100 mass % (QS). The content of water in the structure can be favorably 10 mass % or more, more favorably 25 mass % or more, still more favorably 35 mass % or more, still more favorably 55 mass % or more, still more favorably 65 mass % or more, and favorably 98 mass % or less, more favorably 96 mass % or less, still more favorably 95 mass % or less, still more favorably 94 mass % or less, still more favorably 92 mass % or less. Note that the “content of water in the structure” here includes the water contained in the aqueous phase in the emulsion, in addition to the water in the hydrogel.

### 2.2 Hydrogel

In the present specification, the “hydrogel” forming the continuous phase 11 refers to a gel obtained from a water-soluble polymer gelling agent and water. The “water-soluble polymer gelling agent” refers to a water-soluble organic compound whose aqueous solution obtained by dissolving this in water causes sol-gel transition at the gelling point (freezing point).

In one embodiment, the water-soluble polymer gelling agent refers to at least one selected from a water-soluble non-crosslinked polymer and a water-soluble polymer cross-linked by the addition of metal ions. Specifically, as the water-soluble non-crosslinked polymer, it is favorable to include at least one selected from the group consisting of agar, carrageenan, gellan gum, guar gum, locust bean gum, tara gum, gelatin, xanthan gum, and high methoxyl pectin. These water-soluble non-crosslinked polymers allow excellent disintegrability of the hydrogel structure to be achieved. The non-crosslinked polymers are described in Coombes, A. G. A. et al., “Biocomposites of non-crosslinked natural and synthetic polymers”, Biomaterials, 23(10) (June 2002), pp. 2113-8, which is incorporated herein by reference in its entirety. As the water-soluble polymer cross-linked by the addition of metal ions, for example, it is favorable to include at least one selected from the group of sodium alginate, water-soluble pectin, and carboxymethyl cellulose.

Further, In one embodiment, the water-soluble polymer gelling agent more favorably includes agar. In the present specification, the “agar” refers to a hemicellulose including galactan formed of galactose linked through 1,3 bonds and 1,4 bonds. The agar is formed of two main components of agarose and agaropectin. Agarose is the main component of agar and is a linear polymer having a molecular weight of 20,000 g/mol or more and 1,000,000 g/mol or less, favorably 300,000 g/mol. The molecular weight of agar may be in a range of favorably 20,000 g/mol or more, more favorably 40,000 g/mol or more, still more favorably 50,000 g/mol or more, still more favorably 75,000 g/mol or more, still more favorably 90,000 g/mol or more, and favorably 1,000,000 g/mol or less, more favorably 900,000 g/mol or less, still more favorably 700,000 g/mol or less, still more favorably 500,000 g/mol or less, still more favorably 400,000 g/mol or less. Agarose is a polysaccharide having a structure in which β-D-galactose and 3,6-anhydro-α-L-galactose are alternately linked through α-1,3 bonds and β-1,4 bonds. Agaropectin is a heterogenous mixture of small molecules present in smaller amounts. These structures are similar but slightly divergent, sulfated, and may also have methyl and pyruvate ketal substituent groups. Without being bound by any specific theory, it is conceivable that agarose contributes to mechanical strength of the gel and agaropectin contributes to viscosity. In one embodiment, agar is formed of 50 mass % or more and 90 mass % or less of agarose, favorably 60 mass % or more and 80 mass % or less of agarose. Agar may include favorably 50 weight % or more, more favorably 60 weight % or more of agarose, and may include favorably 90 weight % or less, more favorably 80 weight % or less of agarose. Agar can be regarded as a water-soluble hydrocolloid gelling polymer or a non-crosslinked polymer.

The content of agar in the structure is in a range of favorably 0.1 mass % or more and 5.0 mass % or less, favorably 0.15 mass % or more and 3.0 mass % or less, more favorably 0.2 mass % or more and 2.0 mass % or less, from the viewpoint of providing a gelled structure. The content of agar in the structure may be in a range of favorably 0.1 mass % or more, more favorably 0.15 mass % or more, still more favorably 0.2 mass % or more, and favorably 5.0 mass % or less, more favorably 3.0 mass % or less, still more favorably 2.0 mass % or less.

Further, the water-soluble polymer gelling agent may be added in combination with agar and or more types of different water-soluble non-crosslinked polymers. The mass ratio of agar to the different water-soluble non-crosslinked polymer (the mass of agar/the mass of the different water-soluble non-crosslinked polymer) may be in a range of favorably 1/10 or more and 10 or less, more favorably 1/2 or more and 2 or less. Further, the mass ratio of agar to the different water-soluble non-crosslinked polymer is favorably 1/10 or more, more favorably 1/2 or more, and favorably 10 or less, more favorably 2 or less.

In one embodiment, the hydrogel may contain a thickener. The thickener only needs to have a thickening effect, and can also be expressed as a viscosity adjustor. The thickener include, but not limited to, at least one selected from the group consisting of sodium polyacrylate, polyacrylic acid, a polyacrylic acid copolymer, polyadrylate, polyvinyl alcohol, a maleic anhydride copolymer, an alkyl acrylate copolymer, cross-linked polyacrylic acid, a cross-linked polyacrylic acid copolymer, a cross-linked alkyl acrylic acid copolymer, a cross-linked copolymer of acrylic acid and alkyl acrylate, carboxypolymethylene, carboxymethyl cellulose, sodium carboxymethyl cellulose, sodium polyacrylate starch, sodium acrylate grafted starch, an acrylic acid polymer, a neutralized acrylic acid polymer, a partially neutralized acrylic acid polymer, an acrylate/C10-30 alkyl acrylate crosspolymer, sodium acrylates crosspolymer-2, a sodium acrylate/sodium acryloyldimethyl taurate copolymer, an acrylamide/sodium acryloyldimethyl taurate copolymer, a (sodium polyacrylate/PEG-9 diacrylate) cross-linked polymer, ammonium polyacryloyldimethyl taurate, an ammonium acryloyldimethyl taurate/VP (vinylpyrrolidone) copolymer, a carbomer, and a hydroxyethyl acrylate/sodium acryloyldimethyl taurate copolymer. In some embodiments, the thickener may be povidone, a modified cellulosic type of binder, sodium alginate, carrageenan, a starch-based binder, gum arabic, gellan gum, polydextrose, sorghum malt, or gelatin. Additional thickeners are generally found in Remington's The Science and Practice of Pharmacy, and Handbook of Pharmaceutical Excipients, Arthur H. Kibbe ed. 2000. From the viewpoint of improving the product stability, the hydrogel favorably includes at least one selected from the group consisting of sodium polyacrylate, acrylic acid grafted starch, an acrylate/C10-30 alkyl acrylate crosspolymer, sodium acrylates crosspolymer-2, a (sodium polyacrylate/PEG-9 diacrylate) cross-linked polymer, sodium polyacryloyldimethyl taurate, and a carbomer.

In one embodiment, the content of the thickener in the hydrogel (continuous phase) is favorably 0.001 mass % or more and 0.3 mass % or less, favorably 0.002 mass % or more and 0.2 mass % or less, more favorably 0.003 mass % or more and 0.1 mass % or less, from the viewpoints of reduction of exuded water and handling during mold filling with the solution. The content of the thickener in the structure may be favorably 0.001 mass % or more, more favorably 0.002 mass % or more, still more favorably 0.003 mass % or more, from the viewpoint of reduction of exuded water, and favorably 0.3 mass % or less, more favorably 0.2 mass % or less, still more favorably 0.1 mass % or less, from the viewpoint of handling during mold filling with the solution. In addition, it may be 0.8 mass % or less.

### 2.3 Emulsion

### 2.3.1 Overview

In one embodiment, the emulsion forming the dispersed phase 12 is, for example, one selected from the group consisting of an oil-in-water emulsion, a water-in-oil emulsion, a multiple emulsion, and a nanoemulsion, and is favorably an oil-in-water emulsion, mor favorably an emulsion having a lamellar structure. The oil-in-water emulsion allows favorable usability and functionality to be easily achieved by the synergistic effect with the hydrogel when applied to skin or hair. In the case where the emulsion according to an embodiment is an oil-in-water emulsion, the oil phase 12b is dispersed in the aqueous phase 12a as an emulsified particle as illustrated in FIG. 1. The emulsified particle forming the oil phase 12b favorably has a volume average particle diameter of 0.1 μm or more and 100 μm or less.

### 2.3.1 Oil Phase

The oil phase included in the emulsion can be composed of any compound or an oil agent already generally known or identified in the cosmetics field or dermatological field as being suitable for the manufacture of emulsions, which is physiologically acceptable. In particular, these compounds can be selected, alone or as mixtures, from the group consisting of oils of vegetable, animal, or mineral origin, natural or synthetic waxes, and various other lipid components.

The oil phase usually includes at least one oil. In particular, examples of oils that can participate in the composition of the oil phase include mineral oil, hydrocarbon oil of animal origin, hydrocarbon oil of vegetable origin, synthetic oil, ethers, an alkyl benzoate, a hydrocarbon of minerals or synthetic sources, a fatty alcohol having 8 to 26 carbon atoms, acetylglyceride, a saturated fatty acid, an unsaturated fatty acid, saturated triglyceride, unsaturated triglyceride, silicone oil, and other lipid components. These lipid components can be used alone or in combination of two or more.

Examples of the mineral oil include liquid paraffin, liquid petroleum, and white petroleum.

Examples of the hydrocarbon oil of animal origin include perhydrosqualene and stearic acid. Examples of the hydrocarbon oil of vegetable origin include sweet almond oil, avocado oil, castor oil, coriander oil, olive oil, jojoba oil, sesame oil, peanut oil, canola oil, rapeseed oil, grape seed oil, coconut oil, clary oil, hazelnut oil, shea butter (such as karite butter), cocoa butter, palm oil, apricot kernel oil, calophyllum oil, rice bran oil, corn germ oil, wheat germ oil, soybean oil, sunflower oil, evening primrose oil, safflower oil, passion flower oil, rye oil, stearic acid, and triglycerides of caprylic/capric acids. Note that in the present specification, the term “hydrocarbon (oil)” refers to any oil predominantly including carbon and hydrogen atoms and optionally ester, ether, fluorinated, carboxylic acid, and/or alcohol groups.

Examples of the synthetic oil include purcellin oil, fatty acid esters such as butyl myristate, isopropyl myristate, cetyl myristate, dextrin palmitate, isopropyl palmitate, 2-ethylhexyl palmitate, isopropyl adipate, ethylhexyl adipate, butyl stearate, hexadecyl stearate, and isopropyl stearate, octyl stearate, isocetyl stearate, decyl oleate, hexyl laurate, ethylhexyl isononanoate, propylene glycol dicaprylate, the esters derived from lanolic acid, such as isopropyl lanolate and isocetyl lanolate, dioctylcyclohexane, isoparaffins, and poly-α-olefins;

Examples of the ethers include dicaprylyl ether and cetyl esters. Examples of the cetyl esters include, but are not limited to, favorably cetyl palmitate and cetyl myristate. Examples of the alkyl benzoate include favorably benzoates of C12-C15 fatty alcohols (e.g., Finsolv TN (registered trademark) manufactured by Finetex).

Examples of the hydrocarbon of minerals or synthetic sources include substantially linear or branched hydrocarbons, such as volatile or non-volatile paraffin oils, their derivatives, petroleum jelly, polydecene, isohexadecane, isododecane, and hydrogenated polyisobutene.

Examples of the fatty alcohol having 8 to 26 carbon atoms include lauryl alcohol, cetyl alcohol, myristyl alcohol, stearyl alcohol, palmityl alcohol, oleyl alcohol, linoleyl alcohol, 2-octyldodecanol, and their mixtures. Examples of the mixture include cetearyl alcohol (mixture of cetyl alcohol and of stearyl alcohol).

Examples of the saturated fatty acid include propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, and pentacosylic acid. Of these, the saturated fatty acid favorably includes stearic acid, more favorably stearic acid of non-animal origin.

Examples of the unsaturated fatty acid include α-linolenic acid, stearidonic acid, eicosapentaenoic acid, cervonic acid, linoleic acid, linolelaidic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid.

Examples of the saturated triglyceride include a triglyceride including any of the above saturated fatty acids, favorably caprylic/capric triglycerides. Examples of the unsaturated triglyceride include a triglyceride including at least one unsaturated fatty acid such as those described above.

Silicone oil include volatile or non-volatile silicone oil. Specific examples of silicone oil will be described below.

Examples of the other lipid components include a glyceryl diester, an octanoic acid ester, a decanoic acid ester, a ricinoleic acid ester, fluorinated oil, perfluoro oil, lanolin oil, hydrogenated lanolin, and acetylated lanolin. Examples of the glyceryl diester include one glyceryl diester of the above saturated or unsaturated fatty acid, favorably glyceryl dilaurate. Examples of the octanoic acid ester and the decanoic acid ester include an octanoic acid ester and a decanoic acid ester of an alcohol and a polyhydric alcohol (e.g., glycol and glycerol). Examples of the ricinoleic acid ester include a ricinoleic acid ester of an alcohol and a polyhydric alcohol (e.g., cetyl).

In one embodiment, the oil phase contained in the emulsion include, for example, at least one selected from the group consisting of petrolatum, stearic acid, caprylic/capric triglyceride, a C12-C15 alkyl benzoate, lanolin oil, a cetyl ester, glyceryl dilaurate, a saturated fatty acid, and a saturated triglyceride. In a further embodiment, the saturated fatty acid is stearic acid and the saturated triglyceride is a caprylic/capric triglyceride. In a favorable embodiment, the oil phase includes at least one selected from the group consisting of petrolatum, stearic acid, a caprylic acid/capric triglyceride, C12-C15 alkyl benzoate, lanolin oil, a cetyl ester, and glyceryl dilaurate.

In one embodiment, the content of the oil phase in the structure is in a range of favorably 0.1 mass % or more and 80 mass % or less, favorably 0.5 mass % or more and 50 mass % or less, more favorably 1.0 mass % or more and 40 mass % or less, still more favorably 1.5 mass % or more and 30 mass % or less. The content of the oil phase in the structure may be favorably 0.1 mass % or more, more favorably 0.5 mass % or more, still more favorably 1.0 mass % or more, still more favorably 1.5 mass % or more, and favorably 80 mass % or less, more favorably 50 mass % or less, still more favorably 40 mass % or less, still more favorably 30 mass % or less. Note that the content of the above oil phase refers to the content of one type of oil agent when the oil phase is formed of only the one type of oil agent, and the total content of a plurality of types of oil agents when the oil phase is formed of the plurality of types of oil agents.

### 2.3.2 Amphipathic Molecule

In one embodiment, the emulsion can contain an amphipathic molecule. The amphipathic molecule also functions as an emulsifier and a dispersant, and helps stabilize the emulsion containing the aqueous phase and the oil phase. In one embodiment, examples of the amphipathic molecule include, but not limited to, a fatty acid, a fatty alcohol, polyhydroxy stearic acid, stearic acid, polyethoxylated fatty alcohol, polyglycerolized fatty alcohol (e.g., polyethoxylated stearyl alcohol or cetyl stearyl (cetearyl) alcohol), ceteareth-20, laureth-3, a C16-C30 fatty acid neutralized by an amine, ammonia, or the alkali metal salt thereof, a tertiary amine, an amine oxide, and an amine. Examples of the tertiary amine, the amine oxide, and the amine include an alkyl amine, an alkyl imidazoline, an ethoxylated amine, a quaternary ammonium salt, and a quaternary ester.

Phospholipid emulsifiers can also be used as amphipathic molecule. A phospholipid is a compound that yields, on hydrolysis, phosphoric acid, an alcohol, a fatty acid, and a nitrogenous base. They are widely distributed in nature and include substances such as lecithin, cephalin, and sphingomyelin. Further, alkyl modified silicone emulsifiers may also be used. Examples of the alkyl modified silicone emulsifier include, but not limited to, PEG-9 polydimethylsiloxyethyl dimethicone, PEG/PPG-10/3 oleyl ether dimethicone, and PEG-9 polydimethylsiloxyethyl dimethicone. Examples of other amphipathic molecules include polyoxyethylene hydrogenated castor oil and sorbitan monostearate.

In one embodiment, from the viewpoint of emulsion stability, the content of at least one type of amphipathic molecule in the structure can be favorably 0.05 mass % or more and 6.5 mass % or less, more favorably 0.1 mass % or more and 2.5 mass % or less, still more favorably 0.2 mass % or more and 2 mass % or less. Further, the content of the amphipathic molecule in the structure may be favorably 0.05 mass % or more, more favorably 0.1 mass % or more, still more favorably 0.2 mass % or more, and favorably 6.5 mass % or less, more favorably 2.5 mass % or less, still more favorably 2 mass % or less. In one embodiment, from the viewpoint of emulsion stability, the structure may include two or more types of amphipathic molecules. In this case, the total content of the two or more types of amphipathic molecules in the structure may be favorably 0.5 mass % or more and 6.5 mass % or less, more favorably 1 mass % or more and 4 mass % or less, still more favorably 1.5 mass % or more and 3.5 mass % or less. The total content of the two or more types of amphipathic molecules in the structure may be in a range of favorably 0.5 mass % or more, more favorably 1 mass % or more, more favorably 1.5 mass % or more, and favorably 6.5 mass % or less, more favorably 4 mass % or less, still more favorably 3.5 mass % or less.

In one embodiment, from the viewpoint of enhancing the functionality of the emulsion, the content of the oil phase and the one or more types of amphipathic molecules can be favorably 1 mass % or more and 60 mass % or less, more favorably 2 mass % or more and 50 mass % or less, more favorably 4 mass % or more and 45 mass % or less. The content of the oil phase and the one or more types of amphipathic molecules in the structure may be favorably 1 mass % or more, more favorably 2 mass % or more, still more favorably 4 mass % or more, still more favorably 5 mass % or more, still more favorably 7 mass % or more, still more favorably 10 mass % or more, and favorably 60 mass % or less, more favorably 50 mass % or less, still more favorably 45 mass % or less, still more favorably 35 mass % or less, still more favorably 30 mass % or less, still more favorably 25 mass % or less, still more favorably 20 mass % or less, still more favorably 12 mass % or less.

### 2.3.3 Functional Component of Emulsion

In one embodiment, the emulsion can contain various components having a functionality, and contains, for example, at least one selected from the group consisting of a humectant, a sunscreen agent, and silicone oil.

### 2.3.3.1 Humectant

The humectant includes at least one selected from the group consisting of glycerin, dipropylene glycol, butylene glycol, sorbitol, ethylene glycol, polyethylene glycol, propylene glycol, 1,3-butylene glycol, hexylene glycol, hexanediol, xylitol, erythritol, and mannitol. The humectant favorably includes glycerin from the viewpoint of achieving a high moisturizing effect and pleasant usability with low irritation.

In another embodiment, the humectant may include at least one selected from the group consisting of another polyol, a polysaccharide, sodium hyaluronate, panthenol, urea, hydroxyethyl urea, PEG/PPG/polybutylene glycol-8/5/3 glycerin, hydrolyzed hyaluronic acid, niacinamide, mannose, myristyl malate phosphonic acid, a biosaccharide gum, and combinations thereof.

In one embodiment, from the viewpoint of sufficiently achieving a moisturizing effect, the content of the humectant in the structure can be favorably 0.1 mass % or more and 24 mass % or less, more favorably 2.5 mass % or more and 8.5 mass % or less, still more favorably 3.0 mass % or more and 7.5 mass % or less, still more favorably 3.5 mass % or more and 7.0 mass % or less. The content of the humectant in the structure can be favorably 0.1 mass % or more, more favorably 2.5 mass % or more, still more favorably 3.0 mass % or more, still more favorably 3.5 mass % or more, and favorably 24 mass % or less, more favorably 8.5 mass % or less, still more favorably 7.5 mass % or less, still more favorably 7.0 mass % or less.

### 2.3.3.2 Sunscreen Agent

The sunscreen agent used in one embodiment is also referred to as UV (ultraviolet rays) active or a UV screening agent, and has a function of protecting a surface of skin from damage by ultraviolet rays. The sunscreen agent can be selected from the group consisting of an organic screening agent, a physical screening agent, and their mixtures. In one embodiment, the structure can include, as a sunscreen agent, one or two or more types of UV-A and UV-B screening agents that can be used in the cosmetics field.

Examples of the UV-B screening agent include the following:
(1) salicylic acid derivatives such as homomenthyl salicylate, 2-ethylhexyl salicylate, and triethanolamine salicylate;
(2) cinnamic acid derivatives such as 2-ethylhexyl p-methoxycinnamate, glyceryl di-p-methoxycinnamate mono 2-ethylhexanoate, 2,5-diisopropyl methyl cinnamate, methyl bis(trimethylsiloxy)silylisopentyl trimethoxycinnamate, isopropyl p-methoxycinnamate mixture of isopropyl p-methoxycinnamate and diisopropyl cinnamate, 2-ethoxyethyl p-methoxycinnamate, and diethanolamine p-methoxycinnamate;
(3) liquid β,β′-diphenylacrylate derivatives, in particular 2-ethylhexyl α-cyano-β,β′-diphenylacrylate or octocrylene;
(4) p-aminobenzoic acid derivatives such as p-aminobenzoic acid, glyceryl p-aminobenzoate, ethyl dihydroxypropyl p-aminobenzoate, octyldimethyl p-aminobenzoate, amyl p-dimethyl aminobenzoate, and diethylaminohydroxybenzoyl hexyl benzoate;
(5) 4-methylbenzylidenecamphor;
(6) 2-phenylbenzimidazole-5-sulphonic acid;
(7) 1,3,5-triazine derivatives, in particular 2,4,6-tris[p-(2′-ethylhexyl-1′-oxycarbonyl)anilino]-1,3,5-triazine, and dioctylbutamidotriazone, and 2,4-bis-[{4-(2-ethylhexyloxy)-2-hydroxy}-phenyl]-6-(4-methoxyphenyl)-1,3,5-triazine; and
(8) the mixtures of these screening agents.

Examples of the UV-A screening agent include the following:
(1) dibenzoylmethane derivatives such as 4-isopropyldibenzoylmethane and 4-tert-butyl-4′-methoxydibenzoylmethane;
(2) benzene-1,4-[di(3-methylidenecamphor-10-sulphonic acid)], optionally in the partially or completely neutralized form;
(3) benzophenone derivatives, for example:
   2,4-dihydroxybenzophenone (benzophenone-1);
   2,2′,4,4′-tetrahydroxybenzophenone (benzophenone-2);
   2-hydroxy-4-methoxybenzophenone (benzophenone-3);
   2-hydroxy4-methoxybenzophenone-5-sulphonic acid (benzophenone-4) and its sulphonate form (benzophenone-5);
   2,2′-dihydroxy-4,4′-dimethoxybenzophenone (benzophenone-6);
   5-chloro-2-hydroxybenzophenone (benzophenone-7);
   2,2′-dihydroxy-4-methoxybenzophenone (benzophenone-8);
   the disodium salt of 2,2′-dihydroxy-4,4′-dimethoxybenzophenone-5,5′-disulphonic diacid (benzophenone-9);
   2-hydroxy-4-methoxy-4′-methylbenzophenone (benzophenone-10);
   benzophenone-112-hydroxy-4-(octyloxy)benzophenone (benzophenone-12);
(4) silane derivatives or polyorganosiloxanes including a benzophenone group;
(5) anthranilates, in particular menthyl anthranilate;
(6) compounds including, per molecule, at least two benzazolyl groups or at least one benzodiazolyl group, in particular 1,4-bis(benzimidazolyl)phenylene-3,3′,5,5′-tetrasulphonic acid and its salts;
(7) silicon derivatives of N-substituted benzimidazolyl-benzazoles or of benzofuranyl-benzazoles, for example:
   2-[1-[3-[1,3,3-tetramethyl-1-[(trimethylsilyl)oxy]disiloxanyl]propyl]-1H-benzimidazol-2-yl]benzoxazole;
   2-[1-[3-[1,3,3-tetramethyl-1-[(trimethylsilyl)oxy]disiloxanyl]propyl]-1H-benzimidazol-2-yl]benzothiazole;
   2-[1-(3-(trimethylsilanyl)propyl)-1H-benzimidazol-2-yl]benzoxazole;
   6-methoxy-1,1′-bis(3-(trimethylsilanyl)propyl)-1H, 1′H-[2,2′]benzimidazolyl-benzoxazole;
   2-[1-(3-(trimethylsilanyl)propyl)-1H-benzimidazol-2-yl]benzothiazole; (see Patent Application EP-A-1 028 120);
(8) triazine derivatives and in particular 2,4-bis[4-(2-ethylhexyloxy)-2-hydroxyphenyl]-6-(4-methoxyphenyl)-1,3,5-triazine, 2,2′-methylenebis[6-(2H-benzotriazol-2-yl)-4-(1,1,3,3-tetramethylbutyl)phenol], and 2,4,6-tris[4-(2-ethylhexyloxycarbonyl)anilino]-1,3,5-triazine; and
(9) their mixtures.

In addition to the above-mentioned examples, for example, 2-ethylhexyl dimethoxybenzylidene dioxoimidazolidinepropionate, octocrylene, cinoxate, phenylbenzimidazole sulfonic acid, 1-(3,4-dimethoxyphenyl)-4,4-dimethyl-1,3-pentanedione, 3-(4-methylbenzylidene)camphor, and methylene bis-benzotriazolyl tetramethylbutylphenol can also be used as a sunscreen agent (e.g., an ultraviolet absorber).

It is also possible to use a mixture of several of these screening agents, a mixture of UV-B screening agents and of UV-A screening agents, and also mixtures with physical screening agents.

Examples of the physical screening agent include titanium oxide (amorphous titanium dioxide or crystalline titanium dioxide in the rutile and/or anatase form), zinc oxide, iron oxide, zirconium oxide, cerium oxide, or their mixtures. These metal oxides can be in the form of particles having a micrometric size or nanometric size (nanopigments). In the form of nanopigments, the mean size of the particles is in a range of, for example, 5 nm or more and 100 nm or less.

In a favorable embodiment, the structure further includes at least one sunscreen agent selected from the group consisting of zinc oxide, triethoxycaprylylsilane, titanium dioxide, aluminum hydroxide, stearic acid, butyl methoxydibenzoylmethane, ethylhexyl salicylate, octocrylene, and homosalate.

The content of the at least one sunscreen agent (when a plurality of sunscreen agents is used, the total content of the plurality of sunscreen agents) in the structure is favorably 1 mass % or more and 60 mass % or less, more favorably 5 mass % or more and 50 mass % or less, still more favorably 10 mass % or more and 30 mass % or less. The content of the at least one sunscreen agent in the structure is favorably 1 mass % or more, more favorably 5 mass % or more, still more favorably 10 mass % or more, and favorably 60 mass % or less, more favorably 50 mass % or less, still more favorably 30 mass % or less.

### 2.3.3.3. Silicone Oil

The silicone oil used in one embodiment is used as a texture modifier for improving slipperiness and usability when added in small amounts. However, when sufficiently added as an active ingredient, the silicone oil exhibits functions such as the effects of protecting skin and hair and providing water repellency, in addition to such a texture modification function. In one embodiment, the silicone oil included in the structure is at least one selected from volatile or non-volatile silicone oils. Specifically, examples of the volatile or non-volatile silicone oils include volatile or nonvolatile dimethicone (also known as polymethylsiloxanes, PDMS) with a linear or cyclic silicone chain which are liquid or pasty at ambient temperature, polydimethylsiloxanes including pendent alkyl, alkoxy, or phenyl groups, or alkyl, alkoxy, or phenyl groups at the end of the silicone chain, and phenylated silicone. Examples of dimethicone include particularly cyclopolydimethylsiloxane (cyclomethicone) such as cyclohexasiloxane. Examples of phenylated silicone include phenyl trimethicones, phenyl dimethicones, phenyltrimethylsiloxydiphenylsiloxanes, diphenyl dimethicones, diphenylmethyl-diphenyltrisiloxanes, (2-phenylethyl)trimethylsiloxysilicates, and poly ethylphenylsiloxanes.

In the case where silicone oil is added as an active ingredient, the content of silicone oil in the structure is favorably 0.01 mass % or more and 50 mass % or less, more favorably 0.1 mass % or more and 40 mass % or less, still more favorably 0.5 mass % or more and 30 mass % or less. The content of silicone oil in the structure is favorably 0.01 mass % or more, more favorably 0.1 mass % or more, still more favorably 0.5 mass % or more, and favorably 50 mass % or less, more favorably 40 mass % or less, still more favorably 30 mass % or less. Note that in the case where a plurality of silicone oils is added in combination, the content of the silicone oil is the total content of the plurality of silicone oils.

### 2.3.4 Additive

The structure according to the present disclosure may also contain cosmetically acceptable additives or adjuvants as well as cosmetic or dermatologic active agents. The content of the additive and the adjuvant in the structure can be generally 0.01 mass % or more and 10 mass % or less, favorably 0.05 mass % or more and 5 mass % or less, more favorably 0.1 mass % or more and 3 mass % or less, still more favorably 0.2 mass % or more and 2 mass % or less, still more favorably 0.5 mass % or more and 1 mass % or less. The content of the additive and the adjuvant in the structure may be favorably 0.01 mass % or more, more favorably 0.05 mass % or more, still more favorably 0.1 mass % or more, still more favorably 0.2 mass % or more, and favorably 10 mass % or less, more favorably 3 mass % or less, still more favorably 2 mass % or less, still more favorably 1 mass % or less, still more favorably 0.5 mass % or less. In one embodiment, the content of the additive in the structure is 0.01 mass % or more, 0.05 mass % or more, 0.1 mass % or more, 0.2 mass % or more, 0.3 mass % or more, 0.4 mass % or more, 0.5 mass % or more, 0.6 mass % or more, 0.8 mass % or more, 1 mass % or more, 2 mass % or more, 3 mass % or more, 4 mass % or more, 5 mass % or more, 7 mass % or more, 8 mass % or more, and/or less than 12 mass %, less than 10 mass %, less than 9 mass %, less than 8 mass %, less than 7 mass %, less than 6 mass %, less than 5 mass %, less than 4 mass %, less than 3 mass %, less than 2 mass %, less than 1 mass %, less than 0.8 mass %, less than 0.7 mass %, less than 0.5 mass %, less than 0.3 mass %, less than 0.1 mass %, less than 0.08 mass %, less than 0.05 mass %, less than 0.02 mass %.

Representative additives and adjuvants include, for example, water-soluble or water-miscible solvents or co-solvents, dispersion enhancing agents, moisturizers, reducing sugars such as monosaccharide ketoses, colorants, dyes (including but not limited to Red No. 4, Yellow No. 2, Violet No. 5), pigments, pearlescence agents, inorganic or organic fillers (such as talc, kaolin, silica powder or polyethylene powder), ethanol, preservatives, ionic or nonionic gelling agents and thickening agents, hydrophilic or lipophilic cosmetic or dermatological active principles, antioxidants (e.g., BHT, tocopherol), sequestering agents (e.g., EDTA), essential oils, fragrances, neutralizers, pH-adjusting agents (e.g., triethylamine (TEA) and sodium hydroxide), conditioning or softening agents (e.g., panthenol, allantoin and glycerin), and extracts such as botanical extracts. Examples of cosmetic active agents or dermatological active agents include free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, enhancing agents, menthol, camphor, benzocaine, and salicylic acid. These ingredients may be soluble or dispersible in at least one of the phases present in the structure (i.e., the continuous phase and the aqueous phase and/or oil phase of the dispersed phase).

In one embodiment, the emulsion may contain a thickener. As the thickener, various thickeners described in the section “2.2 Hydrogel” can be used. In some embodiments, the thickener contained in the emulsion favorably includes at least one selected from the group consisting of a carbomer, an acrylate/C10-30 alkyl acrylate cross-linked polymer, and sodium polyacrylate, from the viewpoint of enhancing stability.

Carbomers are thickening cross-linked polymers made of acrylic acid, methacrylic acid and salts thereof. The cross-linking can take place by employing polyfunctional compounds, such as polyaklyl ether of polysaccharides or polyalcohols, such as sucrose allyl ether, pentaerythritol allyl ether, and propylene allyl ether. Homopolymers of acrylic acid or salts thereof, which are cross-linked with a pentaerythritol allyl ether, a sucrose allyl ether, or a propylene allyl ether, are favorable in the context of the present disclosure. A usable thickening agent in the context of the present disclosure is a copolymer including C10-30 acrylic acrylate, acrylic acid, methacrylic acid, and esters thereof, which is cross-linked with a sucrose allyl ether or a pentaerythritol allyl ether. Further, suitable are other known polymeric thickening agents such as Pemulen (registered trademark) polymeric emulsifiers, Noveone (registered trademark) polycarbophils, and Kluce10 (registered trademark). Sodium carboxymethyl cellulose from Aqualon may also be used.

In one embodiment, the carbomer is favorably a neutralized anionic polymer or a neutralized carbomer such as polyacrylic acid (e.g., CARBOPOL (registered trademark) manufactured by Lubrizol Corporation), carboxypolymethylene, and carboxymethyl cellulose, including derivatives of Carbopol (registered trademark) polymers, such as Carbopol Ultrez (registered trademark) 10, Carbopol 940 (registered trademark), Carbopol 941 (registered trademark), Carbopol 954 (registered trademark), Carbopol 980 (registered trademark), Carbopol 981 (registered trademark), Carbopol ETD 2001 (registered trademark), Carbopol EZ-2 (registered trademark), and Carbopol EZ-3 (registered trademark). As used herein, a “neutralized carbomer” is a synthetic, high molecular weight polymer, composed primarily of a neutralized polyacrylic acid. Further, when a base is added to neutralize a carbomer solution, the viscosity of the solution increases.

In one embodiment, the structure may further include an antioxidant. The antioxidant may be at least one selected from the group consisting of citric acid, butylated hydroxytoluene, ascorbic acid, glutathione, retinol, alpha-tocopherol, beta-carotene, alpha-carotene, ubiquinone, butylated hydroxyanisole, ethylenediaminetetraacetic acid, selenium, zinc, lignan, uric acid, lipoic acid, and N-acetylcysteine.

In one embodiment, the structure may further include a reducing sugar. The reducing sugar may be at least one selected from the group consisting of dihydroxyacetone, erythrulose, ribulose, xylulose, galactose, glucose, glyceraldehyde, fructose, ribose, xylose, cellobiose, lactose, maltose, and their mixtures.

In one embodiment, the structure may further include, as an additive, at least one preservative selected from chlorhexidine salts and alkyltrimethylammonium bromides. The chlorhexidine salt may be selected from the group consisting of chlorhexidine digluconate, chlorhexidine diacetate, chlorhexidine hydrochloride, and their mixtures, and the alkyltrimethylammonium bromide may be selected from dodecyltrimethylammonium bromide, myristyltrimethylammonium bromide, hexadecyltrimethylammonium bromide, and their mixtures.

In one embodiment, the structure may be substantially free of specific additives and ingredients. For example, in one embodiment, the structure may be substantially free of or includes a small amount of sodium alginate, scleroglucan, mineral oil, pentane, aliphatic hydrocarbons, hyaluronic acid, polyvinyl alcohol, polyethylene glycol, ethanol, a polysaccharide other than agar, acrylic acid-based polymer, cam extract, aluminum, zinc oxide, or titanium dioxide. Specifically, the content of the above component in the structure can be favorably less than 0.1 mass %, more favorably less than 0.05 mass %, still more favorably less than 0.03 mass %, still more favorably less than 0.01 mass %. In one embodiment, the structure may also include additives other than the above within the upper limit of the above content.

### 3. METHOD OF PRODUCING SOLID HYDROGEL STRUCTURE

### 3.1 Overview

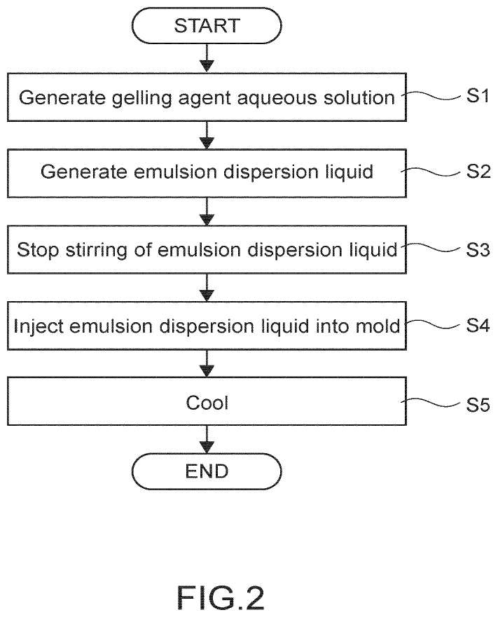

A method of producing a solid hydrogel structure according to another embodiment of the present disclosure includes: a step of generating a gelling agent aqueous solution (S1); a step of generating an emulsion dispersion liquid (S2); a step of stopping stirring of the emulsion dispersion liquid (S3); a step of injecting the emulsion dispersion liquid into a mold (S4); and cooling the emulsion dispersion liquid injected into the mold (S5). This example is shown in FIG. 2.

3.2 S1: Generating Gelling Agent Aqueous Solution

In this step, a gelling agent aqueous solution in which a water-soluble polymer gelling agent is dissolved in water is generated by heating the water-soluble polymer gelling agent and the water to a first temperature range of equal to or higher than a dissolution temperature of the water-soluble polymer gelling agent while stirring them. In one embodiment, the first temperature range is, for example, 50° C. or more and 98° C. or less, favorably 80° C. or more and 98° C. or less, favorably 80° C. or more and 95° C. or less, more favorably 80° C. or more and 90° C. or less, still more favorably 85° C. or more and 89° C. or less, more specifically 85±2° C. Favorably, the water is deionized water, distilled water, purified water, or reverse osmosis water. In one embodiment, the water and the water-soluble polymer gelling agent may be mixed at a temperature of approximately 100° C. The water-soluble polymer gelling agent and the water may be mixed for a time in a range of 10 to 120 minutes, favorably 20 to 90 minutes, more favorably approximately 30 minutes, or until a visibly homogeneous solution is produced. The water-soluble polymer gelling agent and the water may be heated and mixed in a water bath that is heated to the above temperature. In this step, the thickener included in the hydrogel of the continuous phase or other ingredients may be added to the water-soluble polymer gelling agent and the water. Further, the stirring in this step can be performed using a known stirring device used in the art.

### 3.3 S2: Generating Emulsion Dispersion Liquid

In this step, an emulsion dispersion liquid is generated by adding an emulsion containing an oil phase and an aqueous phase to the gelling agent aqueous solution in a second temperature range of a sol-gel transition point of the water-soluble polymer gelling agent and stirring them. In one embodiment, the second temperature range is in a range of 35° C. or more and 60° C. or less, favorably 45° C. or more and 55° C. or less, more favorably 45° C. or more and 52° C. or less. In addition, in the case of emulsion dispersion liquids with high viscosity at low temperatures that are difficult to handle, the second temperature range may be 35° C. or more and 85° C. or less. Cooling the gelling agent aqueous solution from the first temperature range to the second temperature range may be performed in the water bath used in the step S1, may be performed by placing a container containing the gelling agent aqueous solution a water bath or a heating device at a desired temperature different from that in the step S1, or may be performed at room temperature. In one embodiment, the gelling agent aqueous solution may be cooled while stirring to ensure uniform cooling. Further, the emulsion is favorably adjusted to the second temperature range before addition.

The stirring of the emulsion and the gelling agent aqueous solution can be performed for a time in a range of 5 minutes or more and 40 minutes or less, favorably 10 minutes or more and 30 minutes or less, more favorably approximately 15 minutes, or for a time necessary for producing a sufficiently homogeneous emulsion dispersion liquid. Further, the stirring in this step can be performed using a known stirring device used in the art. The stirring device used in this step may be the same stirring device as that in the step S1 or a different stirring device.

Note that the emulsion used in this step may be prepared in advance, or a commercially available skin and/or hair care product may be used. Examples of such commercial products include a moisturizing lotion, a conditioner, balm, hand cream, and an ointment. In the case of using a commercial product, the commercial product itself may be added to the gelling agent aqueous solution, or the commercial product diluted with water may be added. In the latter embodiment, the mass ratio of the commercial product to additional water (mass of commercial product/mass of water) is favorably 1/10 or more and 10 or less, more favorably 1/5 or more and 5 or less, still more favorably 1/2 or more and 2 or less, still more favorably 1. The mass ratio is favorably 1/10 or more, more favorably 1/5 or more, still more favorably 1/2 or more, and favorably 10 or less, more favorably 5 or less, still more favorably 2 or less.

Further, in the case of preparing the emulsion in advance, the aqueous phase and the oil phase are stirred under predetermined temperature conditions and stirring conditions. These temperature conditions and stirring conditions can be appropriately set in accordance with the type of emulsion.

In one embodiment, the emulsion favorably has a phase transition temperature exceeding 50° C. The phase transition temperature of the emulsion here refers to the temperature at which the emulsion type of the emulsion changes. From the viewpoint of maintaining the emulsion, the second temperature range of this step is favorably a temperature equal to or less than the phase transition temperature of the emulsion.

Further, in this step, a water-based dye solution, a flavoring agent, or other additives may be added to the gelling agent aqueous solution.

3.4 S3: Stopping Stirring of Emulsion Dispersion Liquid

In this step, in the second temperature range, the stirring of the emulsion dispersion liquid is stopped. In this step, for example, the operation of the stirring device used in the step S2 is stopped. Note that the temperature in this step only needs to be a temperature included in the second temperature range and may be different from that in the step S2. By stopping the stirring of the emulsion dispersion liquid in the second temperature range of the temperature of the sol-gel transition point or more of the water-soluble polymer gelling agent, stirring during the gelling process is prevented to promote the formation of a continuous phase, and a structure having a continuous phase and a dispersed phase can be molded.

### 3.5 S4: Injecting Emulsion Dispersion Liquid into Mold

In this step, the emulsion dispersion liquid is injected into a mold. From the viewpoint of ease of handling, the mold is favorably formed of a silicone. However, in another embodiment, the mold may be formed of another flexible material such as a thermoplastic elastomer and synthetic rubber. Alternatively, the mold may be formed of a rigid or non-stretching material such as paper, aluminum, or steel. The mold may include wells of individual shapes, such as stars, hearts, bells, squares, and circles to mold the structure into a desired shape. Further, the emulsion dispersion liquid may be directly deposited in a container such as a portion. In this case, the container can be a mold. Further, when performing molding using the container as a mold, the packaging process on the container after cooling can be omitted. The emulsion dispersion liquid may be cooled to a temperature less than the sol-gel transition point to obtain a gelled product, the gelled product may be heated to a temperature equal to or higher than the sol-gel transition point to obtain an emulsion dispersion liquid again, and this emulsion dispersion liquid may be injected into a mold. When the gelled product is heated to obtain an emulsion dispersion liquid again, it only needs to be heated to a temperature equal to or higher than the sol-gel transition point and stirring is not required. Note that the above-mentioned sol-gel transition point means both the temperature at which a sol changes to a gel and the temperature at which a gel changes to a sol, and these temperatures may differ. After obtaining a sol, it is favorable to perform stirring to make the emulsion dispersion liquid uniform.

### 3.6 S5: Cooling Emulsion Dispersion Liquid

In this step, the emulsion dispersion liquid injected into a mold is cooled to a third temperature range of less than the sol-gel transition point. The third temperature range is, for example, a temperature equal to or lower than room temperature, and may be, for example, 2° C. or more and 20° C. less, favorably 4° C. or more and 10° C. or less. In order to sufficiently gelling the emulsion dispersion liquid, it may be cooled while having been injected into a mold for a time such as one day or more in a range capable of sufficiently maintaining quality.

The emulsion dispersion liquid is gelled by being sufficiently cooled, thereby producing a structure according to an embodiment. The gelled structure may be applied to the surface of skin and/or hair as it is or may be packaged.

### 4. ADDITIONAL REMARKS

Although embodiments of the present disclosure have been described, the present disclosure is not limited to only the above-mentioned embodiments, and various modifications can be made without departing from the essence of the present invention.

### 4.1 Another Embodiment of Production Method

### 4.1.1 Overview

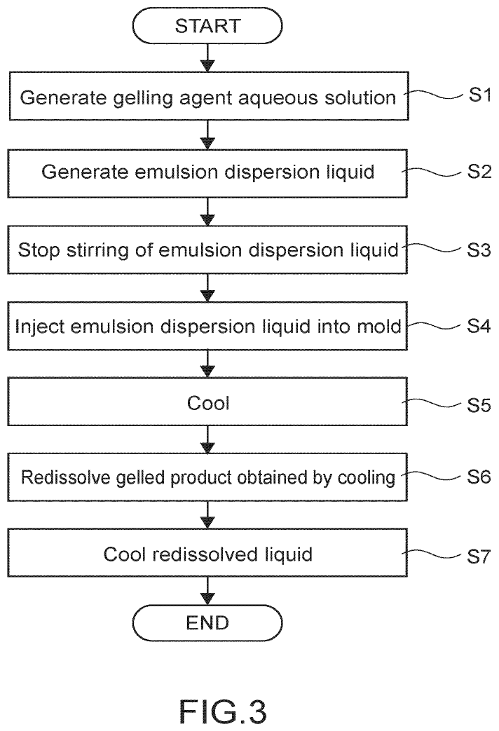

For example, a method of producing a solid hydrogel structure according to another embodiment of the present disclosure further includes, after the step of cooling the emulsion dispersion liquid injected into a mold (S5), a step of redissolving the gelled product obtained by cooling (S6); and a step of cooling the redissolved liquid (S7). This example is shown in FIG. 3. The water-soluble polymer gelling agent used in this embodiment only needs to undergo a reversible sol-gel transition, and favorably includes at least one selected from the group consisting of agar, carrageenan, gellan gum, gelatin, and high methoxyl pectin, more favorably carrageenan from the viewpoint of being capable of suppressing the changes in physical properties due to redissolution because of its low dissolution temperature. Note that in this embodiment, the description of the steps S1 to S5 that overlap with the above-mentioned embodiments is omitted.

### 4.1.2 S6: Step of Redissolving Gelled Product

In this step, the gelled product obtained by cooling is redissolved by heating it to the second temperature range of equal to or higher than the sol-gel transition point of the water-soluble polymer gelling agent again. As a result, the gelled product returns to the state of an emulsion dispersion liquid. The gelled product may be heated in a water bath or may be placed in a heating device to heat it.

### 4.1.3 S7: Step of Cooling Redissolved Liquid

In this step, the redissolved liquid is cooled to the third temperature range of less than the sol-gel transition point while having been injected into the same mold as that in the step S5 or a different mold. The “redissolved liquid” in this step can be an emulsion dispersion liquid, but does not necessarily need to the same physical properties as those of the emulsion dispersion liquid generated in the step S2. For example, the physical properties such as viscosity may differ. In this step, similarly to the step S5, In order to sufficiently gelling the liquid, it may be cooled while having been injected into a mold for a time such as one day or more in a range capable of sufficiently maintaining quality. The redissolved liquid may be contained in the mold into which it was injected in the step S4, throughout the step S5, S6, and S7, and may be added to another container after the step S5 and injected into a mold before the step S7. In the latter case, the mold used in this step may be the mold used in the step S4 or a different mold. In this way, a solid hydrogel structure is formed.

By cooling the redissolved gelled product again to produce a water-soluble hydrogel structure, for example, the formed gelled product can be stored at a temperature higher than the third temperature range and a gelled product can be formed again at arbitrary timing, thereby enhancing the degree of freedom in production.

### 5. EXAMPLES

The following Examples are intended to further describe the protocol for preparing and characterizing a solid hydrogel structure and are not intended to limit the scope of the claims.

### 5.1 Test Example 1: Preliminary Investigation of Physical Properties of Solid Hydrogel Structure

### 5.1.1 Production of Solid Hydrogel Structure

Agar (an example of a water-soluble polymer gelling agent) was dispersed in ion exchanged water at room temperature and heated to 85±10° C. in a water bath for 30 minutes or more and 45 minutes or less while stirring. During the stirring, glycerin was added together with sodium polyacrylate starch if used. After confirming the dissolution of agar, the dissolved agar solution (gelling agent aqueous solution) was cooled to 50±5° C. in a water bath to obtain a warm liquid agar solution. The oil-in-water emulsion in the form of moisturizing lotion was heated to 50±5° C., formulated in the warmed agar solution, and mixed until uniform, thereby generating an emulsion dispersion liquid. This mixture was dispensed into a silicone mold at the processing temperature and cooled at room temperature for a sufficient time to allow easy removal from the mold. Tables 1 to 3 show example formulations. Note that the formulation of the oil-in-water emulsion is shown in Tables 4, 5, 8 described below.

TABLE 1
Example formulation 1

| | | | Prescription Example | | | | |
|---|---|---|---|---|---|---|---|
| Type | Component | Unit | 1 | 2 | 3 | 4 | 5 |
| O/W emulsion | Skin Moisturizer #1 | w/w % | 50.00 | 10.00 | 85.00 | 50.00 | 50.00 |
| O/W emulsion | Skin Moisturizer #2 | w/w % | | | | | |
| O/W emulsion | Sunscreen #1 | w/w % | | | | | |
| Water-soluble polymer gelling agent | Agar | w/w % | 1.50 | 1.50 | 1.50 | 1.50 | 1.50 |
| Humectant | Glycerin | w/w % | 1.00 | 1.00 | 1.00 | 0.10 | 20.00 |
| Thickener | Sodium Polyacrylate Starch | w/w % | | | | | |
| | Water | w/w % | QS | QS | QS | QS | QS |
| | Total | w/w % | 100.00 | 100.00 | 100.00 | 100.00 | 100.00 |
| | Total Humectant | w/w % | 4.38 | 1.68 | 6.74 | 3.48 | 23.38 |
| | Total Water | w/w % | 85.98 | 95.20 | 77.91 | 86.88 | 66.98 |
| | Humectant/Agar Mass Ratio | w/w | 2.92 | 1.12 | 4.49 | 2.32 | 15.58 |
| | Rupture Stress | kPa | 20.53 | 34.75 | 24.03 | 19.33 | 18.88 |
| | Rupture Strain | % | 34.39 | 32.10 | 44.85 | 35.40 | 47.02 |

TABLE 2
Example formulation 2

| | | | Prescription Example | | | | |
|---|---|---|---|---|---|---|---|
| Type | Component | Unit | 6 | 7 | 8 | 9 | 10 |
| O/W emulsion | Skin Moisturizer #1 | w/w % | 50.00 | 50.00 | 50.00 | 50.00 | 50.00 |
| O/W emulsion | Skin Moisturizer #2 | w/w % | | | | | |
| O/W emulsion | Sunscreen #1 | w/w % | | | | | |
| Water-soluble polymer gelling agent | Agar | w/w % | 2.50 | 1.00 | 1.50 | 1.50 | 1.50 |
| Humectant | Propylene Glycol | w/w % | 0.10 | 20.00 | 1.00 | 1.00 | 1.00 |
| Thickener | Sodium Polyacrylate Starch | w/w % | | | 0.10 | 0.10 | 0.50 |
| | Water | w/w % | QS | QS | QS | QS | QS |
| | Total | w/w % | 100.00 | 100.00 | 100.00 | 100.00 | 100.00 |
| | Total Humectant | w/w % | 3.48 | 23.38 | 4.38 | 4.38 | 4.38 |
| | Total Water | w/w % | 85.88 | 67.48 | 85.88 | 85.88 | 85.48 |
| | Humectant/Agar Mass Ratio | w/w | 1.39 | 23.38 | 2.92 | 2.92 | 2.92 |
| | Rupture Stress | kPa | 59.13 | 10.82 | 21.57 | 21.57 | 47.21 |
| | Rupture Strain | % | 34.29 | 42.89 | 40.51 | 40.51 | 69.00 |

TABLE 3
Example formulation 3

| | | | Prescription Example | | |
|---|---|---|---|---|---|
| Type | Component | Unit | 11 | 12 | 13 |
| O/W emulsion | Skin Moisturizer | w/w % | | | |
| | #1 | | | | |
| O/W emulsion | Skin Moisturizer | w/w % | 50.00 | | |
| | #2 | | | | |
| O/W emulsion | Sunscreen #1 | w/w % | | 50.00 | 80.00 |
| Water-soluble | Agar | w/w % | 1.50 | 0.20 | 1.00 |
| polymer gelling | | | | | |
| agent | | | | | |
| Humectant | Glycerin | w/w % | 1.00 | 20.00 | 1.00 |
| Thickener | Sodium | w/w % | 0.15 | | |
| | Polyacrylate | | | | |
| | Starch | | | | |
| | Water | w/w % | QS | QS | QS |
| | Total | w/w % | 100.00 | 100.00 | 100.00 |
| | Total Humectant | w/w % | 3.50 | 21.50 | 3.40 |
| | Total Water | w/w % | 84.81 | 59.30 | 65.20 |
| | Humectant/Agar | w/w | 2.33 | 107.50 | 3.40 |
| | Mass Ratio | | | | |
| | Rupture Stress | kPa | 23.79 | 15.09 | 34.01 |
| | Rupture Strains | % | 55.50 | 35.84 | 44.61 |

### 5.1.2 Evaluation of Rupture Stress

The rupture stress was measured at 25° C. using a TA.XTplusC Texture Analyser (registered trademark) from Stable Micro Systems, United Kingdom. For these measurements, a texture curve of the relationship between compression distance and load was obtained of a sample having dimensions of 25×25×4 mm, using a probe with a diameter of 8 mm. The probe was positioned at the center of a square side of the gel, and during the measurement, the probe had a speed of 2.5 mm/s and the probe was pushed down to 2 mm (probe pushing distance) from the surface. Only if the sample did not break, the probe was pushed down to 3 mm. Four measurements were taken for each sample.

The texture curve obtained showed that the load increases as the compression distance of the hydrogel structure increases, and the load peaks when the hydrogel structure breaks. The rupture stress was obtained from the peak load and compression distance. The obtained breaking load [gf] was divided by the probe area and multiplied by the acceleration of gravity to obtain the rupture stress [kPa].

As shown in Tables 1 to 3, in all of these Prescription Examples, the rupture stress was 8 kPa or more and 65 kPa or less.

### 5.1.3 Example Formulations of Emulsion

Using the emulsion ingredients shown in Table 4 to Table 9, a solid hydrogel structure as an agar gel composition was formed.

TABLE 4
Skin Moisturizer #1

| Ingredient | Content, wt % | Ingredient type |
|---|---|---|
| Deionized Water | Balance (QS) | |
| Glycerin, vegetable grade | 6.75 | Humectant |
| Acrylates/C10-30 Alkyl | 0.07 | Thickener |
| Acrylate Crosspolymer | | |
| Cetearyl Alcohol | 6.2 | Amphiphilic |
| Stearic Acid vegetable grade | | molecule |
| Lanolin Oil | 4 | Oil agent |
| Cetyl Esters NF | | |
| Glyceryl Dilaurate | | |
| Dimethicone | | |
| pH adjuster, fragrance, preservative, | as needed | Additive |
| chelating agent, other additives | (QS) | |
| Total | 100.00 | |

TABLE 5
Skin Moisturizer #2

| Ingredient | Content, wt % | Ingredient type |
|---|---|---|
| Water | Balance (QS) | |
| Glycerin USP | 10.00 | Humectant |
| Carbomer | 0.15 | Thickener |
| Cetearyl Alcohol | 7.5 | Amphiphilic |
| Ceteareth-20 | | molecule |
| Laureth-3 | | |
| Stearic Acid (non-animal derived) | | |
| White Petrolatum USP | 5 | Oil agent |
| C12-15 Alkyl Benzoate | | |
| Dimethicone | | |
| pH adjuster, fragrance, preservative, | as needed | Additive |
| chelating agent, other additives | (QS) | |
| Total | 100.00 | |

TABLE 6
Skin Moisturizer #3

| Ingredient | Content, wt % | Ingredient type |
|---|---|---|
| Deionized Water | Balance (QS) | |
| Glycerin, vegetable grade | 10 | Humectant |
| Acrylates/C10-30 Alkyl | 0.15 | Thickener |
| Acrylate Crosspolymer | | |
| Cetearyl Alcohol | 6.2 | Amphiphilic |
| Stearic Acid vegetable grade | | molecule |
| Lanolin Oil | | Oil agent |
| Cetyl Esters NF | | |
| Glyceryl Dilaurate | | |
| Dimethicone | | |
| Dihydroxyacetone | 0.25-12.0 | Additive |
| pH adjuster, fragrance, | as needed | |
| preservative, chelating agent | (QS) | |
| Total | 100.00 | |

TABLE 7
Skin Moisturizer #4

| Ingredient | Content, wt % | Ingredient type |
|---|---|---|
| Water | Balance (QS) | |
| Glycerin USP | 10 | Humectant |
| Carbomer | 0.15 | Thickener |
| Cetearyl Alcohol | 7.5 | Amphiphilic |
| Ceteareth-20 | | molecule |
| Laureth-3 | | |
| Stearic Acid (non-animal derived) | | |
| White Petrolatum USP | 5 | Oil agent |
| C12-15 Alkyl Benzoate | | |
| Dimethicone | | |
| Dihydroxyacetone | 0.3-13.0 | Additive |
| Erythrulose | 0.02-2.0 | |
| pH adjuster, fragrance, | as needed | |
| preservative, chelating agent | (QS) | |
| Total | 100 | |

TABLE 8
8. Sunscreen #1

| Ingredient | | |
|---|---|---|
| type | Ingredient | Content, wt % |
| | Water | Balance(QS) |
| Humectant | Glycerin USP | 1.6 |
| Thickener | Lauryl Methacrylate/Sodium Methacrylate | 8.5 |
| | Crosspolymer | |
| | Acrylates/C10-30 Alkyl Acrylate | |
| | Crosspolymer | |
| Oil phase | Homosalate | 27.6 |
| | Octocrylene | |
| | Ethylhexyl Salicylate | |
| | Butyl Methoxydibenzoylmethane | |
| | Diethylhexyl 2,6-Naphthalate | |
| | Dextrin Palmitate | |
| | Dicaprylyl Ether | |
| | Phenyl Trimethicone | |
| Amphiphilic | Glyceryl Behenate | 0.7 |
| molecule | Cetyl Alcohol NF | |
| | Sorbitan Distearate | |
| | Dextrin Palmitate | |
| | Stearoyl Glutamic Acid | |
| Additive | pH adjuster, fragrance, | as needed (QS) |
| | preservative, chelating agent | |
| | Total | 100 |

Table 9 illustrates a prescription of a sunscreen formula, having mineral agents for protecting against UV damage.

TABLE 9
9. Sunscreen #2

| Type | Ingredient | Content, wt % |
|---|---|---|
| | Water | Balance (QS) |
| Humectant | Dipropylene glycol | 5 |
| Thickener | Hydroxyethyl Acrylate/Sodium | 0.5 |
| | Acrylcyldimethyl Taurate Copolymer | |
| | (or) Carbomer (or) Acrylates/ | |
| UV active | Zinc Oxide | 10-36 |
| | Titanium Dioxide | 1-15 |
| Oil agent | Ethylhexyl Isononanoate | 5-10 |
| | C12-15 Alkyl Benzoate | 1.5 |
| | Dextrin Palmitate | 0.5 |
| Dispersant | Polyhydroxystearic Acid | 0.1-1 |
| | pH adjusters, fragrance, preservatives, | as needed |
| | chelating agents. other additives | (QS) |

### 5.2 Test Example 2: Investigation of Functionality of Solid Hydrogel Structure

### 5.2.1 Production of Sample

### 5.2.1.1 Example A1

A solid hydrogel structure according to Example A1 was formed in the same manner as that in Test Example 1. Specifically, agar (an example of a water-soluble polymer gelling agent) was dispersed in ion exchanged water at room temperature and heated to 85±2° C. in a water bath while stirring. It was stirred at this temperature for approximately 30 minutes, the resolution of agar was confirmed, and then, the dissolved agar solution (gelling agent aqueous solution) was cooled to 50±2° C. in a water bath, thereby obtaining a warm liquid agar solution. The O/W emulsion prepared by stirring the aqueous phase and the oil phase was heated to approximately 50° C., formulated in the warmed gelled aqueous solution, and stirred until uniform, thereby generating an emulsion dispersion liquid. As necessary, a humectant, a flavoring agent, other components were added together with the O/W emulsion. Next, this mixture was dispensed into a silicone mold at approximately 50° C. and cooled at room temperature for approximately 30 minutes. After that, the solidified gelled product was carefully taken out of the mold, thereby obtaining a solid hydrogel structure.

The formulation in Example A1 is shown in Table 10. In Example A1, as an emulsion, a moisturizing lotion-like emulsion included in the 0 phase and the W phase of the O/W emulsion shown in Table 10 was added. The sol-gel transition point of the water-soluble polymer gelling agent (agar in this case) was 42° C. Further, the stirring stop temperature of the emulsion dispersion liquid was set to 50° C. as shown in Table 10,

### 5.2.1.2 Comparative Example A1

A sample according to Comparative Example A1 was formed in the same manner as that in Example A1 except that the stirring stop temperature of the emulsion dispersion liquid was changed to 30° C. as shown in Table 10 and then the mixture was dispensed into a silicone mold. However, the sample according to Comparative Example A1 had no specific shape and was amorphous.

### 5.2.1.3 Comparative Example A2

A sample according to Comparative Example A2 was generated in the same manner as that in Example A1 except that agar was not used. However, the sample according to Comparative Example A2 was not gelled and was liquid.

### 5.2.1.4 Example B1

A solid hydrogel structure according to Example B1 was formed in the same manner as that in Example A1 except that an emulsion including a sunscreen agent was used as an emulsion.

### 5.2.1.5 Comparative Example B1

A sample according to Comparative Example B1 was generated in the same manner as that in Example B1 except that agar was not used. However, the sample according to Comparative Example B1 was not gelled and was liquid.

TABLE 10

| | | | | Example | Comparative | Comparative | Examine | Comparative |
|---|---|---|---|---|---|---|---|---|
| | Ingredient type | INCI Name | | A1 | Example A1 | Example A2 | B1 | Example B1 |
| Continuous | Water-soluble polymer | Agar | [wt. %] | 1.50 | 1.50 | | 1.20 | |
| phase | gelling agent | | | | | | | |
| | Water | Water | [wt. %] | 48.50 | 48.50 | 50.00 | 28.80 | 30.00 |
| | Total continuous phase | | [wt. %] | 50.00 | 50.00 | 50.00 | 30.00 | 30.00 |
| O/W | Amphiphilic molecule | C [symbol] Alcohol | [wt. %] | 2.05 | 2.05 | 2.05 | 0.46 | 0.46 |
| emulsion | Amphiphilic molecule | Stearic Acid | [wt. %] | 1.08 | 1.08 | 1.08 | 0.22 | 0.22 |
| O | Sunscreen agent(UV filter) | A [symbol] benzene | [wt. %] | | | | 3.00 | 3.00 |
| phase | Sunscreen agent(UV filter) | E [symbol] S [symbol] late | [wt. %] | | | | 5.00 | 5.00 |
| | Sunscreen agent(UV filter) | Homo [symbol] ate | [wt. %] | | | | 10.00 | 10.00 |
| | Sunscreen agent(UV filter) | Oct [symbol] | [wt. %] | | | | 5.00 | 5.00 |
| | Sunscreen agent(UV | Diethylhexyl | [wt. %] | | | | 1.50 | 1.50 |
| | boo [symbol] ) | 2, [symbol] -Naphthalate | | | | | | |
| | | Dextr [symbol] P [symbol] | [wt. %] | | | | 1.00 | 1.00 |
| | Silicone oil | D [symbol] | [wt. %] | 0.30 | 0.30 | 0.30 | | |
| | Total O/W emulsion oil phase | | [wt. %] | 3.43 | 3.43 | 3.43 | 26.17 | 26.17 |
| O/W | Humectant | [symbol] | [wt. %] | 3.38 | 3.38 | 3.38 | | |
| emulsion | Thickener | Acrylates/C10-30 Alkyl | [wt. %] | 0.04 | 0.04 | 0.04 | 0.50 | 0.50 |
| W | | Acrylate Crosspolymer | | | | | | |
| phase | Thickener | Sodium Acrylates | [wt. %] | 0.40 | 0.40 | 0.40 | | |
| | | Crosspolymer-2 | | | | | | |
| | pH adjuster | Sodium Hydroxide | [wt. %] | 0.08 | 0.06 | 0.08 | 0.11 | 0.11 |
| | Water | Water | [wt. %] | 42.71 | 42.71 | 42.71 | 43.22 | 43.22 |

| | | | | | | |
|---|---|---|---|---|---|---|
| Total O/W emulsion aqueous phase | [wt. %] | 46.58 | 46.58 | 46.58 | 43.83 | 43.83 |

| | | | | | | |
|---|---|---|---|---|---|---|
| Total O/W emulsion | [wt. %] | 50.00 | 50.00 | 50.00 | 70.00 | 70.00 |
| Total | [wt. %] | 100.00 | 100.00 | 100.00 | 100.00 | 100.00 |
| Sol-gel transition temperature of water-soluble polymer | [° C.] | 42 | 42 | — | 42 | |
| Stirring stop temperature of emulsion dispersion liquid | [° C.] | 50 | 30 | 50 | 50 | 50 |
| Rupture Stress | [kPa] | 20 | — | — | 29 | — |
| TEW [symbol] | [g/m [symbol] ] | 10 | — | 10 | — | — |
| Conductance | [—] | 100 | — | 70 | — | — |
| SPF | [—] | — | — | — | 26 | 20 |

[symbol]

indicates data missing or illegible when filed

### 5.2.2 Evaluation of Rupture Stress

The rupture stress was measured in the same manner as that in Test Example 1. The results are shown in Table 10.

As shown in Table 10, the rupture stress in both Example A1 and Example B1 was within the range of 8 kPa or more and 65 kPa or less. Meanwhile, in Comparative Example A1 in which the stirring of the water-soluble polymer gelling agent was performed until below the sol-gel transition point, a solid hydrogel structure could not be formed and rupture stress could not be measured. Similarly, in the liquid samples according to Comparative Example A2 and Comparative Example B1, rupture stress could not be measured.

### 5.2.3 Evaluation of Functionality

### 5.2.3.1 Preparation of Subject

For (five) subjects, the lower limb of the measuring site was cleaned with cotton soaked in an acetone/diethyl ether equal amount mixture and then exposed to outside air for at least 10 minutes. After that, approximately 1 g of each of the samples according to Example A1 and Comparative Example A2 was applied to the measurement site (forearm) of each subject to prepare for measuring transepidermal water loss (TEWL) and conductance.

### 5.2.3.2 Measurement Environment

The measurements were conducted under the conditions of an ambient temperature of 20 to 22° C. and a relative humidity of 40 to 60%. Before the measurements, the subjects were acclimatized to the measurement environment for at least 15 to 30 minutes.

### 5.2.3.3 Method of Measuring Transepidermal Water Loss (TEWL)

Transepidermal water loss (TEWL) was measured using AquaFlux (registered trademark) Model AF200 TEWL Measurement Device from Biox Systems Ltd. The device was turned on for at least 30 minutes before measurement and stabilized, and then, the measurement was conducted. TEWL was measured 5 times at the sample application site of the subject, and the average value thereof was used as the result.

### 5.2.3.4 Method of Measuring Conductance (Stratum Corneum Electric Resistance)

Conductance (stratum corneum electric resistance) was measured as an index of the stratum corneum moisture content. The conductance was measured using Hydration Pin Probe mounted in DermaLab Combo from CortexTechnology. The device was turned on for at least 30 minutes before measurement and stabilized, and then, the measurement was conducted. The measurement was conducted 5 times at the sample application site of the subject, and the average value thereof was used as the result.

### 5.2.3.5 Method of Measuring SPF

Each of the samples according to Example B1 and Comparative Example B1 was uniformly applied onto a square PMMA plate at a concentration of 1.4 mg/cm2 for 1 minute and dried in a cool and dark place for 15 minutes. After drying the sample, the absorption spectra were measured at a total of nine points, i.e., the center point, respective vertices, and midpoints of the sides connecting the vertices of the square PMMA plate, using UV-2000S Ultraviolet Transmittance Analyzer manufactured by Labsphere. The average value of the nine points was used as an SPF value.

### 5.2.3.6 Results of TEWL and Conductance

As shown in Table 10, the TEWL of the site to which Example A1 that is a solid hydrogel structure was applied was lower than TEWL of the site to which Comparative Example A2 that is a general liquid moisturizing lotion was applied. Further, the conductance of the site to which Example A1 that is a solid hydrogel structure was applied was higher than the conductance of the site to which Comparative Example A2 that is a general liquid moisturizing lotion was applied. From these results, it was found that the solid hydrogel structure according to Example A1 is capable of improving the function of moisturizing skin by reducing the water evaporation from skin as compared with the general moisturizing lotion.

### 5.2.3.7 Results of SPF

As shown in Table 10, the SPF of the site to which Example B1 that is a solid hydrogel structure was higher than the SPF of the site to which Comparative Example B1 that is a general liquid lotion was applied. From this result, it was found that the solid hydrogel structure according to Example B1 is capable of improving the sunscreen function as compared with Comparative Example B1 that is a general lotion-type sunscreen agent. This is presumably because Example B1 that is a solid hydrogel structure is likely to stay on the application site and the effect of the sunscreen agent at the application site is sufficiently exhibited.

### 5.3 Test Example 3: Evaluation of Physical Properties of Variety of Examples

As Test Example 3, samples according to a plurality of Examples using a humectant, an ointment including silicone oil, and a sunscreen agent were prepared and the physical properties were evaluated.

### 5.3.1 Production of Samples

Samples of solid hydrogel structures according to Examples A2 to A11, Example C1, and Examples B2 to B3 shown in Table 11 were produced in the same manner as that in Test Example 1. A humectant was used as an emulsion in Examples A2 to A11, an ointment including silicone oil (dimethicone) was used as an emulsion in Example C1, and a sunscreen agent was used as an emulsion in Examples B2 to B3. Further, the sol-gel transition point of the water-soluble polymer gelling agent (agar in this case) was 42° C., and the stirring stop temperature of the emulsion dispersion liquid was 50° C.

TABLE 11

| | | | Exam- | Exam- | Exam- | Exam- | Exam- | Exam- | Exam- |
|---|---|---|---|---|---|---|---|---|---|
| | Ingredient type | INCI Name | ple A2 | ple A3 | ple A4 | ple A5 | ple A6 | ple A7 | ple A8 |
| Continuous | Water-soluble polymer | Agar | 1.50 | 1.50 | 1.50 | 1.50 | 1.50 | 2.50 | 1.00 |
| phase | gelling agent | | | | | | | | |
| | | Glycerin | 1.00 | 1.00 | 1.00 | 0.10 | 20.00 | 0.10 | 20.00 |
| | | Sodium Acrylates | | | | | | | |
| | | Crosspolymer-2 | | | | | | | |
| | Water | Water | 47.50 | [symbol] 7.50 | 12.50 | 48.40 | 28.50 | 47.40 | 29.00 |
| | Total continuous phase | | 50.00 | [symbol] 0.00 | 15.00 | 50.00 | 50.00 | 50.00 | 50.00 |
| O/W | Amphiphilic molecule | Myristyl Alcohol | | | | | | | |
| emulsion | Amphiphilic molecule | Cetyl Alcohol | | | | | | | |
| O | Amphiphilic molecule | Cetearyl Alcohol | 2.05 | 0.41 | 3.49 | 2.05 | 2.05 | 2.05 | 2.05 |
| phase | Amphiphilic molecule | Stearyl Alcohol | | | | | | | |
| | Amphiphilic molecule | Stearic Acid | 1.08 | 0.22 | 1.03 | 1.08 | 1.08 | 1.08 | 1.08 |
| | Amphiphilic molecule | [symbol] | | | | | | | |
| | | L [symbol] | | | | | | | |
| | | C [symbol] -20 | | | | | | | |
| | | Glyceryl [symbol] | | | | | | | |
| | | Sorbitan Distearate | | | | | | | |
| | | S [symbol] G [symbol] Acid | | | | | | | |
| | Sunscreen agent(UV filter) | A [symbol] benzene | | | | | | | |
| | Sunscreen agent(UV filter) | Ethylhexyl Salicylate | | | | | | | |
| | Sunscreen agent(UV filter) | Homo [symbol] ate | | | | | | | |
| | Sunscreen agent(UV filter) | Octorylene | | | | | | | |
| | Sunscreen agent(UV boo [symbol] ) | Di [symbol] | | | | | | | |
| | | 2, [symbol] -Naphthalate | | | | | | | |
| | | D [symbol] P [symbol] | | | | | | | |
| | | Peb [symbol] | | | | | | | |
| | Silicone oil | Di [symbol] | 0.30 | 0. [symbol] 9 | [symbol] .32 | 0.30 | 0.30 | 0.30 | 0.30 |
| Total O/W emulsion oil phase | | | 3.43 | 0. [symbol] | 11.14 | 3.43 | 3.43 | 3.43 | 3.43 |
| O/W | Humectant | Glycerin | 3.38 | 0. [symbol] | [symbol] .74 | 3.38 | 3.38 | 3.38 | 3.38 |
| emulsion | Thickener | Acrylates/C10-30 | 0.04 | 0.01 | 0.06 | 0.04 | 0.04 | 0.04 | 0.04 |
| W | | Alkyl Acrylate | | | | | | | |
| phase | | Crosspolymer | | | | | | | |
| | Thickener | Acrylates/C10-30 | | | | | | | |
| | | Alkyl Acrylate | | | | | | | |
| | | Crosspolymer | | | | | | | |
| | Thickener | Sodium Acrylates | 0.40 | 0.0 [symbol] | 0. [symbol] | 0.40 | 0.40 | 0.40 | 0.40 |
| | | Crosspolymer-2 | | | | | | | |
| | | [symbol] | | | | | | | |
| | Thickener | Poly [symbol] -3 [symbol] | | | | | | | |
| | pH adjuster | Sodium Hydroxide | 0.06 | 0.01 | 0.10 | 0.06 | 0.06 | 0.06 | 0.06 |
| | pH adjuster | Lactic Acid | | | | | | | |
| | Water | Water | 42.71 | 6. [symbol] 0 | [symbol] | 42.71 | 42.71 | 42.71 | 42.71 |

| | | | | | | | |
|---|---|---|---|---|---|---|---|
| Total O/W emulsion aqueous phase | 4 [symbol] .5 [symbol] | 9.38 | [symbol] 3.87 | 48.58 | 48.58 | 48.58 | 49.58 |

| | | | | | | | |
|---|---|---|---|---|---|---|---|
| Total O/W emulsion | 50.00 | 10.00 | [symbol] 5.00 | 50.00 | 50.00 | 50.00 | 50.00 |
| Total | 100.00 | 100.00 | 100.00 | 100.00 | 100.00 | 100.00 | 100.00 |
| Sol-gel transition temperature of water-soluble polymer | 42 | 42 | 42 | 42 | 42 | 42 | 42 |
| Stirring stop temperature of emulsion dispersion liquid | 50 | 50 | 50 | 50 | 50 | 50 | 50 |
| Rupture Stress | 21 | 35 | 24 | 1 [symbol] | 1 [symbol] | [symbol] | 11 |
| SPF | — | — | — | — | — | — | — |
| Stability | | | | | | | |
| Ease of crumbling | 5.0 | 4.2 | 4.8 | 5.0 | 5.0 | 3.0 | 3.2 |
| Ease of application | 5.0 | 4.8 | 4.2 | 5.0 | 4.0 | 5.0 | 4.8 |

| | | | Exam- | Exam- | Exam- | Exam- | Exam- | Exam- |
|---|---|---|---|---|---|---|---|---|
| | Ingredient type | INCI Name | ple A9 | ple A10 | ple A11 | ple C1 | ple B2 | ple B3 |
| Continuous | Water-soluble | Agar | 1.50 | 1.50 | 1. [symbol] 0 | 1.20 | 0.20 | 1.00 |
| phase | polymer | | | | | | | |
| | gelling agent | | | | | | | |
| | | Glycerin | 1.00 | 1.00 | 1.00 | | 20.00 | 1.00 |
| | | Sodium Acrylates | 0.20 | 0.50 | 0.15 | | | |
| | | Crosspolymer-2 | | | | | | |
| | Water | Water | 47.50 | 47.50 | 47.50 | 28.80 | 29.80 | 18.00 |
| | Total continuous phase | | 50.00 | 50.00 | 50.00 | 30.00 | 50.00 | 20.00 |
| O/W | Amphiphilic molecule | Myristyl Alcohol | | | | 1.85 | | |
| emulsion | Amphiphilic molecule | Cetyl Alcohol | | | | 0.23 | 0.0 [symbol] | 0.15 |
| O | Amphiphilic molecule | Cetearyl Alcohol | 2.05 | 2.05 | 2.30 | | | |
| phase | Amphiphilic molecule | Stearyl Alcohol | | | | 0. [symbol] | | |
| | Amphiphilic molecule | Stearic Acid | 1.08 | 1.08 | 1.08 | | | |
| | Amphiphilic molecule | [symbol] | | | | 0.50 | | |
| | | L [symbol] | | | 0.25 | | | |
| | | C [symbol] -20 | | | 0.13 | | | |
| | | Glyceryl [symbol] | | | | | 0.14 | 0.22 |
| | | Sorbitan Distearate | | | | | 0.08 | 0.12 |
| | | S [symbol] G [symbol] Acid | | | | | 0.03 | 0.05 |
| | Sunscreen agent(UV filter) | A [symbol] benzene | | | | | 1.54 | 2.48 |
| | Sunscreen agent(UV filter) | Ethylhexyl Salicylate | | | | | 2.50 | 4.00 |
| | Sunscreen agent(UV filter) | Homo [symbol] ate | | | | | 5.00 | 8.00 |
| | Sunscreen agent(UV filter) | Octorylene | | | | | 2.50 | 4.00 |
| | Sunscreen agent(UV boo [symbol] ) | Di [symbol] | | | | | 1.00 | 1. [symbol] 0 |
| | | 2, [symbol] - | | | | | 0.51 | 0.82 |
| | | Naphthalate | | | | | | |
| | | D [symbol] P [symbol] | | | 1.50 | | | |
| | | Peb [symbol] | | | | | | |
| | Silicone oil | Di [symbol] | 0.30 | 0.30 | 0. [symbol] | 20.00 | | |
| | Total O/W emulsion oil phase | | 3.43 | 3.43 | 5.53 | 23.13 | 13.38 | 21.41 |
| O/W | Humectant | Glycerin | 3.38 | 3.38 | 5.00 | 20.00 | 0.7 [symbol] | 1.21 |
| emulsion | Thickener | Acrylates/C10-30 | 0.04 | 0.04 | | | | |
| W | | Alkyl Acrylate | | | | | | |
| phase | | Crosspolymer | | | | | | |
| | Thickener | Acrylates/C10-30 | | | | | 0.20 | 0.32 |
| | | Alkyl Acrylate | | | | | | |
| | | Crosspolymer | | | | | | |
| | Thickener | Sodium Acrylates | 0.40 | 0.40 | | | | |
| | | Crosspolymer-2 | | | | | | |
| | | [symbol] | | | 0.05 | | | |
| | Thickener | Poly [symbol] -3 [symbol] | | | | 0.4 [symbol] | | |
| | pH adjuster | Sodium Hydroxide | 0.06 | 0.06 | 0.12 | | 0.0 [symbol] | 0.07 |
| | pH adjuster | Lactic Acid | | | | 0.18 | | |
| | Water | Water | 42.71 | 42.71 | 30.30 | 28.27 | 3 [symbol] .61 | 58.98 |

| | | | | | | |
|---|---|---|---|---|---|---|
| Total O/W emulsion aqueous phase | 45.58 | 4 [symbol] | 44.47 | 46.87 | 36. [symbol] | 5 [symbol] . [symbol] |

| | | | | | | |
|---|---|---|---|---|---|---|
| Total O/W emulsion | 50.00 | 50.00 | 50.00 | 70.00 | [symbol] 0.00 | 80.00 |
| Total | 100.00 | 100.00 | 100.00 | 100.00 | 100.00 | 100.00 |
| Sol-gel transition temperature of water-soluble polymer | 42 | 4 [symbol] | 4 [symbol] | 4 [symbol] | 4 [symbol] | 4 [symbol] |
| Stirring stop temperature of emulsion dispersion liquid | 50 | 50 | 50 | 50 | 50 | 50 |
| Rupture Stress | 22 | 47 | 24 | 23 | 15 | [symbol] 4 |
| SPF | — | — | — | — | | |
| Stability | | | | Stable | | |
| Ease of crumbling | 5.0 | 3. [symbol] | 4. [symbol] | 4.9 | 4.2 | 4.2 |
| Ease of application | 5.0 | 3.4 | 3. [symbol] | 3.0 | 5.0 | 4.2 |

[symbol]

indicates data missing or illegible when filed

### 5.3.2 Evaluation of Physical Properties

### 5.3.2.1 Evaluation of Rupture Stress

The rupture stress of each of the samples according to Examples A2 to A11, Example C1, and Examples B2 to B3 was measured in the same manner as that in Test Example 1. As shown in Table 11, the rupture stress of each of the samples according to these Examples was 8 kPa or more and 65 kPa or less.

### 5.3.2.2 Measurement and Evaluation of Separation Stability

40 g of the sample according to Example C1 was placed in a glass bottle, stored in an environment of 50° C. for one week, and then, the presence or absence of separation of the emulsion in the sample was visually evaluated. As a result, in the sample according to Example C1, the emulsion was not separated and maintained the stable state.

The silicone oil (dimethicone) included in Example C1 is known to easily separate from the aqueous phase. It is known that when separation stability is evaluated similarly for the emulsion including approximately 20 mass % of dimethicone, the emulsion is separated. Meanwhile, it was found that in the sample according to Example C1, the separation of the emulsion is suppressed to improve the stability because the emulsion is encapsulated in the gel structure formed by agar.

### 5.3.2.3 Evaluation of Ease of Crumbling

A panelist took 1 g of each of the samples according to Examples A2 to A11, Example C1, and Examples B2 to B3 in his/her hand and applied to the skin of the forearm, and the ease of crumbling during application was evaluated. The number of panelist was five, they evaluated the samples on a 5-point scale (1 point or more to 5 points), and the average score of the panelists was used as the result. Regarding the specific evaluation, 1 point indicates that it is difficult to crumble, 2 points indicate that it is somewhat difficult to crumble, 3 points indicate that it crumbles without any problems, 4 points indicate that it is easy to crumble, and 5 points indicate that it is very easy to crumble. Samples with 3 points or more were evaluated to “Pass”.

As shown in Table 11, all of the samples according to Examples A2 to A11, Example C1, and Examples B2 to B3 had 3 points or more, and it was found that they crumble by hand without any problems.

### 5.3.2.4 Evaluation of Ease of Application

A panelist took 1 g of each of the samples according to Examples A2 to A11, Example C1, and Examples B2 to B3 in his/her hand and applied to the skin of the forearm, and the ease of application during application was evaluated. The number of panelist was five, they evaluated the samples on a 5-point scale (1 point or more to 5 points), and the average score of the panelists was used as the result. Regarding the specific evaluation, 1 point indicates that it is difficult to apply, 2 points indicate that it is somewhat difficult to apply, 3 points indicate that it applies without any problems, 4 points indicate that it is easy to apply, and 5 points indicate that it is very easy to apply. Samples with 3 points or more were evaluated to “Pass”.

As shown in Table 11, all of the samples according to Examples A2 to A11, Example C1, and Examples B2 to B3 had 3 points or more, and it was found that they can be applied to skin without any problems.

### 5.4 Test Example 4: Evaluation of Physical Properties for Examples of Variety of Sunscreen Agents

As Test Example 4, a plurality of samples using a variety of sunscreen agents was further prepared and the physical properties were evaluated.

### 5.4.1 Production of Samples

### 5.4.1.1 Example D1, Example E1, Example F1, and Example G1

Solid hydrogel structures according to Example D1, Example E1, Example F1, and Example G1 were formed in the same manner as that in Example A1 of Test Example 1 except that an emulsion including a sunscreen agent was used as an emulsion. The content of ingredients included in Example D1 is shown in Table 12. The content of ingredients included in each of Example E1, Example F1, and Example G1 is shown in Table 13.

However, the emulsion used in Example D1 was prepared as follows. The respective ingredients shown in Table 12 were mixed at the amounts shown in Table 12 while heating to 80° C. Water heated to 80° C. was added thereto, thereby producing an emulsion. The emulsion was homogenized and then cooled to 40° C.

In Example E1, Zinc Oxide (CI 77947) treated with OTS or dimethicone was used.

### 5.4.1.2 Example D2

A solid hydrogel structure according to Example D2 was formed in the same manner as that in Example D1 except that carrageenan was used instead of agar and dissolved at 60° C. The content of the ingredients included in Example D2 is shown in Table 12.

### 5.4.1.3 Example D3

A solid hydrogel structure was formed in the same manner as that in Example D2, and then, this solid hydrogel structure was allowed to stand at room temperature to cool, heated to 60±2° C. in a water bath and allowed to stand for one hour, and maintained at 60±2° C. in the water bath and stirred for one hour, thereby preparing a redissolved liquid sample. Next, this redissolved sample was dispensed into a silicone mold again and cooled at room temperature for approximately 30 minutes. After that, the solidified gelled product was carefully taken out of the mold, thereby obtaining a solid hydrogel structure according to Example D3. The content of the ingredients included in Example D3 is shown in Table 12.

### 5.4.1.3 Comparative Example D1

A sample according to Comparative Example D1 was generated in the same manner as that in Example D1 except that a water-soluble polymer gelling agent such as agar was not used. The sample according to Comparative Example D1 was not gelled and was liquid. The content of the ingredients included in Comparative Example D1 is shown in Table 12.

TABLE 12

| | | | | | Example | Example | Example | Comparative |
|---|---|---|---|---|---|---|---|---|
| | Type | Ingredient | | | D1 | D2 | D3 | Example D1 |
| Continuous | Water-soluble | Agar | Agar | [wt. %] | 3.1 | | | |
| phase | polymer gelling agent | | | | | | | |
| | Water-soluble | Carrageenan | Carrageenan | [wt. %] | | 1.7 | 1.7 | |
| | polymer gelling agent | | | | | | | |
| | Thickener | Starch sodium | Sodium acrylate | [wt. %] | 0.5 | 0.5 | 0.5 | 0.5 |
| | | acrylate polymer | grafted starch | | | | | |
| | Water | Water | Water | [wt. %] | 26.4 | 27.8 | 27.8 | 29.5 |
| | Total continuous phase | | | [wt. %] | 30.0 | 30.0 | 30.0 | 30.0 |
| Emulsion | Sunscreen agent | Ethylhexyl | Ethylhexyl | [wt. %] | 11.0 | 11.0 | 11.0 | 11.0 |
| oil | (UV absorber) | Methoxycinnamate | Methoxycinnamate | | | | | |
| phase | Sunscreen agent | Bis-Ethylhexyloxyphenol | Bis-Ethylhexyloxyphenol | [wt. %] | 2.0 | 2.0 | 2.0 | 2.0 |
| | (UV absorber) | Methoxyphenyl Triazine | Methoxyphenyl Triazine | | | | | |
| | Sunscreen agent | Diethylamino | Diethylamino | [wt. %] | 2.0 | 2.0 | 2.0 | 2.0 |
| | (UV absorber) | Hydroxybenzoyl | Hydroxybenzoyl | | | | | |
| | | Hexyl Benzoate | Hexyl Benzoate | | | | | |
| | Sunscreen agent | Ethylhexyl Triazone | Ethylhexyl Triazone | [wt. %] | 1.0 | 1.0 | 1.0 | 1.0 |
| | (UV absorber) | | | | | | | |
| | Base | C12-15 Alkyl Benzoate | C12-15 Alkyl Benzoate | [wt. %] | 4.0 | 4.0 | 4.0 | 4.0 |
| | Amphiphilic molecule | Glyceryl Behenate | Glyceryl Behenate | [wt. %] | 2.0 | 2.0 | 2.0 | 2.0 |
| | Amphiphilic molecule | Cetyl Alcohol | Cetanol | [wt. %] | 1.0 | 1.0 | 1.0 | 1.0 |
| | Amphiphilic molecule | Sorbitan Distearate | Sorbitan Distearate | [wt. %] | 1.0 | 1.0 | 1.0 | 1.0 |
| | Total emulsion oil phase | | | [wt. %] | 24.0 | 24.0 | 24.0 | 24.0 |
| | Water | Water | Water | [wt. %] | 46.0 | 46.0 | 46.0 | 46.0 |

| | | | | | |
|---|---|---|---|---|---|
| Total emulsion aqueous phase | [wt. %] | 46.0 | 46.0 | 46.0 | 46.0 |

| | | | | | |
|---|---|---|---|---|---|
| Total emulsion | [wt. %] | 70.0 | 70.0 | 70.0 | 70.0 |
| Total | [wt. %] | 100.0 | 100.0 | 100.0 | 100.0 |
| Sol-gel transition | [° C.] | 42 | 35 | 35 | — |
| temperature of | | | | | |
| water-soluble polymer | | | | | |
| Dissolution operation | [° C.] | 85 | 60 | 60 | — |
| temperature of | | | | | |
| gelling agent | | | | | |
| Stirring stop temperature | [° C.] | 50 | 50 | 50 | 50 |
| of bulk solution | | | | | |
| Redissolution operation | | — | — | — | — |
| Rupture Stress | [kPa] | 12.0 | 21.5 | 20.2 | — |
| SPF | [—] | 31 | 36 | 28 | 11 |
| Ease of crumbling | [—] | 4.8 | 5.0 | 5.0 | — |
| Ease of application | [—] | 4.6 | 4.8 | 4.6 | — |

TABLE 13

| | | | | | Example | Example | Example |
|---|---|---|---|---|---|---|---|
| | Type | Ingredient | | | E1 | F1 | G1 |
| Continuous | Water-soluble | Agar | Agar | [wt. %] | 1.00 | [symbol] .0 | 1. [symbol] 2 |
| phase | polymer gelling agent | | | | | | |
| | Water | Water | Water | [wt. %] | 19.00 | 1 [symbol] .0 | 1 [symbol] . [symbol] |
| | Total continuous phase | | | [wt. %] | [symbol] | [symbol] | 20.00 |
| | Sunscreen agent (UV absorber) | [symbol] | [symbol] | [wt. %] | [symbol] .00 | 0. [symbol] | 2.40 |
| | Sunscreen agent (UV absorber) | [symbol] | [symbol] | [wt. %] | [symbol] .00 | 2.0 | |
| | Sunscreen agent (UV absorber) | [symbol] | [symbol] | [wt. %] | [symbol] .00 | 2.0 | |
| | Sunscreen agent (UV absorber) | Ethylhexyl Salicylate | Ethylhexyl Salicylate | [wt. %] | 4.00 | 4.0 | |
| | Sunscreen agent (UV absorber) | [symbol] | [symbol] | [wt. %] | | 2.0 | |
| | Sunscreen agent | Zinc Oxide ( [symbol] ) | Zinc Oxide | [wt. %] | [symbol] .00 | | 080 |
| | (UV [symbol] ) | | | | | | |
| | Sunscreen agent | T [symbol] Dioxide ( [symbol] ) | [symbol] Oxide | [wt. %] | | [symbol] .0 | |
| | (UV [symbol] ) | | | | | | |
| | Base | C12-C15 A [symbol] | C12-C15 A [symbol] | [wt. %] | 1.00 | | |
| | Base | [symbol] | Glyceryl [symbol] | [wt. %] | | [symbol] | 4.00 |
| | Base | [symbol] | [symbol] | [wt. %] | | | 4.00 |
| | Base | [symbol] | [symbol] | [wt. %] | | | 2. [symbol] |
| | Base | [symbol] | [symbol] | [wt. %] | | | 0.80 |
| | Base | [symbol] | [symbol] | [wt. %] | | | 0.80 |
| | Base | [symbol] | [symbol] | [wt. %] | | 0.3 | |
| | Base | Stearic Acid | Stearic Acid | [wt. %] | | 0.1 | |
| | Base | [symbol] | [symbol] | [wt. %] | | 0.1 | |
| | Base | Silica | Silica | [wt. %] | | 0.1 | |
| | Thickener | [symbol] | [symbol] | [wt. %] | | 0. [symbol] | |
| | Thickener | [symbol] | [symbol] | [wt. %] | | 4.0 | |
| | [symbol] | PEG-20 Di [symbol] | PEG- [symbol] | [wt. %] | | 2.0 | |
| | [symbol] | [symbol] | [symbol] | [wt. %] | | 0. [symbol] | |
| | [symbol] | [symbol] | [symbol] | [wt. %] | | | 2.40 |
| | Total emulsion oil phase | | | [wt. %] | [symbol] .00 | [symbol] | [symbol] 0 |
| Emulsion | [symbol] | Silica | Silica | [wt. %] | 4.00 | | |
| aqueous | [symbol] | P [symbol] | P [symbol] | [wt. %] | 0.0 [symbol] | | |
| phase | UV absorber | Phenyl [symbol] Acid | Phenyl [symbol] Acid | [wt. %] | | | 2. [symbol] 0 |
| | [symbol] | [symbol] | [symbol] | [wt. %] | | | 1. [symbol] 0 |
| | Thickener | C [symbol] | C [symbol] | [wt. %] | | | 0. [symbol] |
| | Viscosity [symbol] | [symbol] Gum | Xanthan Gum | [wt. %] | | | 0. [symbol] |
| | Emulsifier | [symbol] | [symbol] | [wt. %] | | | 1. [symbol] |
| | Emulsifier | [symbol] | [symbol] | [wt. %] | | | 0. [symbol] |
| | Wetting agent | Glycerin | Glycerin | [wt. %] | | | 1. [symbol] |
| | Wetting agent | Dipropylene Glycol | Dipropylene Glycol | [wt. %] | | | 1. [symbol] |
| | Wetting agent | [symbol] Glycol | [symbol] Glycol | [wt. %] | | | 1. [symbol] |
| | [symbol] agent | Di [symbol] | [symbol] | [wt. %] | | | 0. [symbol] |
| | Base | [symbol] | [symbol] | [wt. %] | | | [symbol] |
| | [symbol] | P [symbol] | P [symbol] | [wt. %] | | | 0. [symbol] |
| | Water | Water | Water | [wt. %] | [symbol] . [symbol] | 2 [symbol] | 4 [symbol] |

| | | | | |
|---|---|---|---|---|
| Total emulsion aqueous phase | [wt. %] | [symbol] | [symbol] | [symbol] |

| | | | | |
|---|---|---|---|---|
| Total emulsion | [wt. %] | [symbol] .00 | [symbol] | [symbol] |
| Total | [wt. %] | 100.00 | 100.0 | 100.00 |
| Sol-gel transition | [° C.] | 4 [symbol] | 4 [symbol] | 4 [symbol] |
| temperature of | | | | |
| water-soluble polymer | | | | |
| Dissolution operation | [° C.] | 85 | [symbol] | [symbol] |
| temperature of gelling agent | | | | |
| Stirring stop temperature | [° C.] | [symbol] 0 | [symbol] | 5 [symbol] |
| of bulk solution | | | | |
| Redissolution operation | | — | — | — |
| Rupture Stress | [kPa] | [symbol] | [symbol] | [symbol] |
| SPF | [—] | [symbol] | [symbol] | [symbol] |
| Ease of crumbling | [—] | [symbol] | [symbol] | [symbol] |
| Ease of application | [—] | [symbol] | [symbol] | [symbol] |

[symbol]

indicates data missing or illegible when filed

### 5.4.2 Evaluation of Physical Properties

### 5.4.2.1 Evaluation of Rupture Stress

The rupture stress was measured for the samples according to Examples D1 to D3, Example E1, Example F1, and Example G1 in the same manner as that in Test Example 1. As shown in Tables 12 and 13, the rupture stress of each of the samples according to these Examples was 8 kPa or more and 65 kPa or less. The rupture stress could not be measured for the liquid sample according to Comparative Example D1.

### 5.4.2.2 Evaluation of Ease of Crumbling

As in Test Example 3, a panelist took 1 g of each of the samples according to Examples D1 to D3, Example E1, Example F1, and Example G1 in his/her hand and applied to the skin of the forearm, and the ease of crumbling during application was evaluated.

As shown in Tables 12 and 13, all of the samples according to Examples D1 to D3, Example E1, Example F1, and Example G1 had 3 points or more, and it was found that they crumble by hand without any problems. The ease of crumbling could not be evaluated for the liquid sample according to Comparative Example D1.

### 5.4.2.3 Evaluation of Ease of Application

As in Test Example 3, a panelist took 1 g of each of the samples according to Examples D1 to D3, Example E1, Example F1, and Example G1 in his/her hand and applied to the skin of the forearm, and the ease of application during application was evaluated.

As shown in Tables 12 and 13, as in Test Example 3, all of the samples according to Examples D1 to D3, Example E1, Example F1, and Example G1 had 3 points or more, and it was found that they can be applied to skin without any problems.

### 5.4.2.4 Measurement and Evaluation of SPF

For the samples according to Examples D1 to D3, Comparative Example D1, Example E1, Example F1, and Example G1, the SPF was measured in the same manner as that in Test Example 2. As shown in Table 12, the SPF of the site to which each of Examples D1 to D3 that are solid hydrogel structures was applied was higher than the SPF of the site to which Comparative Example D1 that is liquid lotion including no hydrogel was applied. From this result, it was found that the solid hydrogel structure according to each of Examples D1 to D3 is capable of improving the sunscreen function as compared with Comparative Example D1 including no hydrogel. This is presumably because each of Examples D1 to D3 that are solid hydrogel structures is likely to stay on the application site and the effect of the sunscreen agent at the application site is sufficiently exhibited. Further, as shown in Table 13, it was confirmed that Example E1, Example F1, and Example G1 had the sunscreen function.

### REFERENCE SIGNS LIST

10 solid hydrogel structure (structure)
11 continuous phase
12 dispersed phase
12a aqueous phase
12b oil phase

### INDUSTRIAL APPLICABILITY

According to the present disclosure, it is possible to provide a solid hydrogel structure that has a specific shape and is capable of exhibiting high functionality by being applied to skin and/or hair, and a method of producing the same.

1. A solid hydrogel structure, comprising:
a continuous phase that is formed of a hydrogel comprising a water-soluble polymer gelling agent and water; and
a dispersed phase that is formed of an emulsion comprising an aqueous phase and an oil phase, and
wherein the dispersed phase is dispersed in the continuous phase.

2. The solid hydrogel structure according to claim 1, wherein the solid hydrogel structure has rupture stress of 8 kPa or more and 65 kPa or less.

3. The solid hydrogel structure according to claim 1, wherein a mass ratio of the dispersed phase to the continuous phase is 10/90 or more and 90/10 or less.

4. The solid hydrogel structure according to claim 1, wherein the dispersed phase has a dimeter of 0.001 μm or more and 1000 μm or less.

5. The solid hydrogel structure according to claim 1, wherein the emulsion comprises an amphipathic molecule.

6. The solid hydrogel structure according to claim 1, wherein the emulsion is an oil-in-water emulsion.

7. The solid hydrogel structure according to claim 1, wherein the emulsion comprises at least one selected from the group consisting of a humectant, a sunscreen agent, and silicone oil.

8. The solid hydrogel structure according to claim 1, wherein the water-soluble polymer gelling agent comprises at least one selected from the group consisting of agar, carrageenan, gellan gum, guar gum, locust bean gum, tara gum, gelatin, xanthan gum, and high methoxyl pectin.

9. The solid hydrogel structure according to claim 1, wherein the hydrogel comprises a thickener.

10. A method of producing a solid hydrogel structure, the method comprising:
generating a gelling agent aqueous solution in which a water-soluble polymer gelling agent is dissolved in water by heating the water-soluble polymer gelling agent and the water to a first temperature range of equal to or higher than a dissolution temperature of the water-soluble polymer gelling agent while stirring them;
generating an emulsion dispersion liquid by adding an emulsion containing an oil phase and an aqueous phase to the gelling agent aqueous solution in a second temperature range of a sol-gel transition point of the water-soluble polymer gelling agent and stirring them;
stopping the stirring of the emulsion dispersion liquid in the second temperature range;
injecting the emulsion dispersion liquid into a mold; and
cooling the emulsion dispersion liquid injected into the mold to a third temperature range of less than the sol-gel transition point.

11. The method of claim 10, wherein the first temperature range is 50° C. or more and 98° C. or less.

12. The method of claim 10, wherein the second temperature range is 35° C. or more and 85° C. or less.

13. The method of claim 10, wherein the emulsion has a phase transition temperature of exceeding 50° C.

14. The method of claim 10, further comprising:
cooling the emulsion dispersion liquid to the third temperature range and then heating the gelled product obtained by cooling to the second temperature range to redissolve the gelled product; and
cooling the redissolved liquid to the third temperature range while the redissolved liquid has been injected into the same mold or a different mold.
